(19) United States
(12) Patent Application Publication
(10) Pub. No.: US 2006/0124770 A1
(43) Pub. Date: Jun. 15, 2006

(54) Spray nozzle and mixing block

(75) Inventors: Daniel C. Venard, Centennial, CO (US); Benjamin A. Young, Colorado Springs, CO (US); Todd Leifheit, Littleton, CO (US)

Correspondence Address: SHERIDAN ROSS PC, 1560 BROADWAY, SUITE 1200, DENVER, CO, 80202, US

(73) Assignee: Castle Rock Industries, Inc., Englewood, CO (US)

(21) Appl. No.: 11/352,191

(22) Filed: Feb. 9, 2006

Related U.S. Application Data
(63) Continuation of application No. 11/253,100, filed on Oct. 17, 2005.
Application No. 11/253,100 is a continuation-in-part of application No. 11/059,663, filed on Feb. 15, 2005.
Application No. 11/059,663 is a continuation-in-part of application No. 10/737,027, filed on Dec. 15, 2003.
Application No. 10/737,027 is a continuation-in-part of application No. 10/438,485, filed on May 14, 2003.

(51) Int. Cl. B05B 11/06; A61M 11/02; B05B 7/04; A62C 11/00

(52) U.S. Cl. 239/398; 239/366; 239/368; 239/361

(57) ABSTRACT

The present invention relates generally to a spray nozzle that is configured to mix at least two cleaning solutions and then to apply the mixed cleaning solutions to a surface to be cleaned. Furthermore, the spray nozzle can be configured to mix the at least two cleaning solutions at various ratios, thereby creating a customized cleaning solution for each type of surface that is to be cleaned.

### CROSS-REFERENCE TO RELATED APPLICATIONS

This application is a Continuation of U.S. patent application Ser. No. 11/253,100, filed Oct. 17, 2005, which is a Continuation-in-Part of U.S. patent application Ser. No. 11/059,663 filed Feb. 15, 2005, which is a Continuation-in-Part of U.S. patent application Ser. No. 10/737,027, filed Dec. 15, 2003, which is a Continuation-in-Part of U.S. patent application Ser. No. 10/438,485, filed May 14, 2003, all of which are incorporated by reference in their entirety herein.

### FIELD OF THE INVENTION

The present invention relates to an spray nozzle for use in cleaning various surfaces. More specifically, the spray nozzle is capable of mixing and metering solutions in an open environment just before application to a surface to be cleaned.

### BACKGROUND OF THE INVENTION

Cleaning machines are used extensively for cleaning flooring surfaces comprised of tile, stone, brick, wood, concrete, carpets and other common surfaces. Maintaining the cleanliness of these surfaces, especially in high volume areas in commercial, industrial, institutional and public buildings is an ongoing and time consuming process. The present invention relates to a highly maneuverable floor cleaning or treatment apparatus (hereinafter “treatment apparatus”) that supports an operator during use. More specifically, some embodiments of the present invention are adapted to clean, sweep, vacuum, burnish, wax, etc. (hereinafter “treat”) a floored surface, wherein the operator is supported by the cleaning device, thus increasing efficiency and productivity of the cleaning operation. As used herein, “floored surface”, or more generally “surface”, encompasses areas covered by concrete, tile, carpet, wood, plastic, stone, turf or any other substance known in the art. The prior devices address many issues that arise with cleaning such floored surfaces. Unfortunately, prior to the present invention, there was no one device that could address many, if not all, of the issues that arise in cleaning various surfaces in various environments at any given point in time.

A. Mop And Bucket Cleaning Devices

In the past, building maintenance staff and others often treat surfaces, such as tiled hallways or restroom floors, using traditional mop and bucket techniques. The bucket may include a detachable mop ringer and may be positioned on caster wheels to facilitate easy movement. Depending on the cleanliness of the equipment, a worker may be able to make a good start in treating a floor using the mop and bucket approach. However, soon the mop and fluid in the bucket becomes soiled or otherwise contaminated by germs and/or bacteria. From that point on, each time the worker plunges the mop into the bucket and rings the mop, both the mop and cleaning fluid become more and more dirty/contaminated.

B. Manually-Propelled Cleaning Devices

The basic cleaning problems associated with the prior art mop & bucket approach to cleaning a surfaces have generally been addressed in the art, as shown in U.S. Pat. No. 6,206,980 to Robinson, entitled “Multi-Functional Cleaning Machine,” which is fully incorporated herein by reference. This type of cleaning machine generally includes a manually propelled wheeled body with two tanks, one concentrated chemical receptacle, a vacuum and blower motor and a fluid pumping system. Typically, such equipment includes only a single motor used for both vacuuming soiled fluid and blowing air that can be used to dry a cleaned surface. While such equipment is generally maneuverable and is an improvement over the earlier mop and bucket technology, the system is still labor intensive and slow. As a result, productivity of cleaning professionals, when using these type of systems is generally decreased over what it might be with other type of systems that are available.

C. Self-Propelled Walk-Behind Device

Productivity concerns have been addressed in the art by the creation of certain walk behind floor treatment apparatus. These apparatus typically have a scrub deck at machine's front and a squeegee at its rear. The squeegee has the ability to “swing” or follow the path of the scrub deck as the machine changes direction. This type of equipment is generally more efficient in cleaning large surface areas than either the mop and bucket or the manually propelled devices. Unfortunately, however, the distance between the scrub deck and squeegee is relatively great. Also, walkbehinds typically have relatively wide squeegees. These characteristics limit such machine's maneuverability and limit the doorways they can easily pass through. Typical 3′ doorway allows a machine with no more than a 33″ squeegee to fit through without removal.

Small walk behind floor cleaning apparatus typically include a scrub deck in the middle of the machine and squeegees at the machine's rear. In this configuration the squeegee has little or no ability to swing or follow the path of the scrub deck as the machine changes direction. Small rider scrubbers typically have relatively narrow squeegees, and rely on “side squeegees” (unvacuumized squeegee blades) adjacent to the scrub deck to direct the water into the path of the main (vacuumized) squeegee. The problem with these side squeegees is that they do not perform very well for very long and tend to leave a film of water in turns because the vacuumized squeegee does not follow the true path of the scrub deck, only the path of the side squeegees (which leave the film of water). Finally, side squeegee are typically very heavy rubber blades and have significant down-pressure applied to them to direct the water—this makes them expensive and causes significant “drag” which increases the work for the propel unit and limits battery run-time. Thus, while more maneuverable than larger walk behind floor treatment machines, the small machines typically do not clean as well as the larger machines.

D. Storage Issues in Prior Art Devices

Further, known cleaning machines do not provide adequate onboard storage for cleaning supplies, tools, etc. Likewise, prior art machines do not often provide a flexible approach to adding storage facilities for trash and the like when the need for such arises. Machinery that addresses these issues is therefore needed.

E. Self-Propelled Ride-On Devices

Self-propelled cleaning devices are generally also well known in the field and are employed to treat large floored surfaces, such as tiled, concrete or carpeted floors found in hospitals, department stores, schools, gyms, etc. These devices generally provide the operator with seating from which he/she can control operation of the device. These devices are ideal for cleaning large, open areas because they are capable of containing large amounts of waste fluids and/or debris without having to repeatedly perform time consuming fluid replacement or debris removal. Moreover, because these devices provide the user with seating, the user does not become prematurely fatigued, increasing overall worker productivity. Unfortunately, these large ride-on machines are not particularly well-suited for cleaning smaller, more confined floor surfaces, which are often found in hallways, small rooms, or even large rooms which have many obstacles therein.

As is well known in the art, smaller self-propelled cleaning devices are also in existence that are ideal for cleaning the smaller rooms and hallways. However, smaller devices are usually pushed or pulled by an operator. Hence, the major drawback of these devices is that they often rely on operator strength to maneuver the device. Even if the device is self-propelled, it often employs manual steering. After a long shift of walking behind a treatment device, the operator is bound to become fatigued, wherein his or her attention will deviate from the task at hand, thereby possibly resulting in uneven treatment to the floored area. Thus, a subsequent crew may have to return and retouch certain areas that were not accurately treated during the first operation. In addition, human errors related to the amount of time a surface is exposed to a brush, may occur when the operator lingers over a single area for extended period of time. This situation is never good for a floor surface. The devices in the art are also difficult to maneuver and often are not adapted to operate around tight corners, wherein pre or post cleaning operations must be performed, thus increasing the time and expense of the entire task.

Thus, it is a long felt need in the field of floor cleaning or treatment to provide a device that allows the operator to ride thereon, and which is adapted to be used in small areas and/or around tight corners. The following disclosure describes an improved floor cleaning and treatment device that is adapted for use in small areas that includes a platform adapted to support the operator to ensure optimum floor cleaning or treatment.

### SUMMARY OF THE INVENTION

It is one aspect of the present invention to provide a floor treatment apparatus that is easy to maneuver. More specifically, one embodiment of the present invention is constructed of a chassis section that includes an enclosure that houses at least a portion of the internal components of the treatment device and a location for installation of devices that are used during cleaning operations. In addition, one embodiment of the present invention provides a standing, leaning or sitting location for the operator. Another embodiment of the present invention is equipped with a powered steering device that allows for greater maneuverability in areas with tight corners, thereby ensuring that more of the flooring surface is treated without having to perform pre or post treatment operations. More specifically, one embodiment of the present invention is equipped with a self-propelled wheel and an easy to use steering device to provide increased maneuverability around obstacles. One embodiment of the present invention employs at least one wheel that provides thrust and/or steering capability. Yet another embodiment of the present invention employs wheels that are substantially centered under the chassis such that the entire apparatus is generally capable of 360° rotation without substantially traversing in any other direction, thus allowing it to treat tight corners of a surface. It is another aspect of the present invention to provide a cleaning apparatus that is cost effective to manufacture. Various aspects of the invention shall now be described in more detail.

Chassis

One embodiment of the present invention employs a chassis section that is designed to protect and house the internal workings of the apparatus and provide a location for interconnection of auxiliary treatment devices used therewith. One embodiment of the present invention employs a chassis that is constructed of rigid plastic, metal, or other common materials used in the art. The chassis of this embodiment also is equipped with a platform for the operator. Alternative embodiments of the present invention employ a foldable, removable or stationary operator seat. In addition, other safety features such as pads or belts may be employed to secure the operator into the cleaning device and thus his/her working environment.

It is yet another aspect of the present invention to provide a chasis with a small envelope. More specifically, one embodiment of the present invention is small enough to fit into and through tight spaces. Often facilities that employ the apparatus of the present invention include narrow door ways, aisles and elevators. In addition, especially in older buildings that have been retrofitted to comply with the Americans with Disabilities Act, elevators are of minimal volume and lifting capability. To fit into small elevators, the chassis is designed to have the smallest practical envelope, a distinct advantage over the prior art. Also, the apparatus of one embodiment of the present invention includes components that are easily removable or adjustable to reduce the profile of the apparatus. Thus, the embodiments of the present invention may be used in various structures.

Steering Mechanism

Another aspect of the present invention is to provide a cleaning apparatus that is easy to operate and maneuver. More specifically, one embodiment of the present invention is equipped with a steering mechanism that allows for inputs from the operator to be efficiently communicated to the steering wheels of the cleaning apparatus. Alternatively, other steering means may be used to facilitate maneuverability of the treatment apparatus, such as joy sticks, touch screens, buttons, remote control elements, etc.

It is still yet another aspect of the present invention to provide a cleaning apparatus that is adapted to efficiently clean areas with tight corners. More specifically, one embodiment of the present invention is adapted to generally perform 360° turns without appreciable lateral motion. This embodiment of the present invention is equipped with a turning mechanism generally under the center of the chassis with two powered exterior wheels adjacent thereto that provide power to the chassis to pivot around the centered wheel. The powered exterior wheels may be independently controlled by joy sticks, wherein movement thereof send directional inputs to each wheel. One embodiment of the invention is equipped with at least one joy stick wherein forward deflection will impart forward motion, rearward deflection will impart rearward motion, and a side-to-side deflection will cause the apparatus to turn. Alternatively, two joy sticks may be used in a similar manner, wherein rearward deflection of the left joy stick and forward deflection of the right joy stick will result in a left turn, and depending on the placement of the powered wheels, perhaps a 360° left hand turn.

Another embodiment of the present invention utilizes a steering wheel, handle bars, a yoke, or similar apparatus for steering. Embodiments may also include a power-assisted steering mechanism.

Power Plant

It is another aspect of the present invention to provide a treatment apparatus that is powered by commonly used power plants. More specifically, one embodiment of the present invention employs an electric motor to power the apparatus. The electric motor may be powered by batteries, solar energy or an electrical cord attached to a permanent power source. Alternatively, the present invention may be powered by an internal combustion engine. Other propulsion means may also be employed by the present invention without departing from its scope, as will be appreciated by one skilled in the art.

Floor Treatment Devices

One embodiment of the present invention employs a chassis that houses a fluid pump assembly and a vacuum assembly. The apparatus further includes at least two tanks, one for retaining a base cleaning fluid, such as water, and a second for retaining spent cleaning solution, dry debris, etc. The apparatus may also include one or more concentrated cleaning chemical receptacles designed to hold concentrated cleaning chemicals. The receptacles are preferably stored within a lockable structure, adding safety to the overall apparatus. These agents can be added to a base cleaning fluid just prior to application to a surface and as desired to facilitate cleaning of various surfaces.

In accordance with one embodiment of the invention, two tanks may be used. A first tank may carry the base fluid and a second tank may be used as a recovery tank for collecting dirty recovery fluid. An inventive aspect of the present invention provides an integrated first and second tank interface. The interface between the tanks is configured such that when separated, the opening of the base fluid tank acts like a funnel. When the base fluid tank needs to be refilled with water for instance, a user can pour the solution into the tank and the amount of spillage normally encountered by completing such an action is reduced due to the shape of the base fluid tank.

Tanks

As briefly mentioned above, preferably at least one tank is provided that provides a solution that is directed towards the flooring surface to be cleaned to facilitate treatment. The tank may be constructed with multiple compartments wherein waste water from the surface is contained prior to disposal. More specifically, one embodiment of the present invention employs a tank that includes a movable membrane. In this configuration, the clean water and/or cleaning solution is deposited on a surface and agitated. Dirty water is next suctioned up and deposited back into a portion of the tank, thereby moving a membrane accordingly to accept the dirty water. Such a configuration is disclosed in U.S. Pat. No. 4,759,094, which is herein incorporated in its entirety by this reference. A similar selectively expandable fluid storage area can be created by utilizing a collapsible structure, which is placed inside of the primary fluid tank. This type of arrangement is disclosed in U.S. Pat. No. 4,196,492, which is also incorporated herein in its entirety by this reference.

Clean water can obviously come from an outside source such as a hose, rather than be stored on board the device. However, in order to facilitate maneuverability and usability of the present invention, it is envisioned that the chassis will house or hold at least one fluid tank and perhaps a plurality thereof.

Cleaning Solutions

In one type of treatment operation, fluid from the chemical receptacles flows through a tube to a chemical selector, which may include a metering valve. The selector preferably has a positive shut-off position, wherein fluid is prevented from flowing through the selector regardless of the fluid pressure in a fluid line. The selector is responsive to input from an operator selection of one of the several cleaning chemicals. Once a chemical is selected, it is free to flow through the chemical selector and appropriate amounts thereof may be provided to one of any number of inlets to a mixing tee. The amount of chemical allowed to flow may be adjusted by a metering valve built into the selector or separate from the selector, in a known fashion. A base cleaning fluid, such as water, may flow from a fluid tank and through a separate tube to a second leg of a mixing tee. The cleaning fluid and concentrated cleaning chemical then mix within the mixing tee to create a cleaning solution. That solution may then be passed through the selector outlet to a pressure pump, wherein the cleaning solution may be pressurized and communicated via appropriate tubing to a dispensing device. The pump, which draws fluid to and through the selector, also preferably includes a bypass system to facilitate regulation of pump pressure. Use of the pump to draw fluid is preferred as it does not create unwanted pressures in the fluid lines and the system, in general, is not subject to gravity feeding of fluid.

A solution may be applied to a surface using any type of dispensing device. In a preferred embodiment, the dispensing device or associated solution lines or tubes include an adjustable valve, which may be used to adjust the pressure and flow of solution allowed to exit the dispensing device. Because of the adjustability, the apparatus may be utilized as a pre-cleaner for various carpet treatments, including spotting or other treatments.

By use of the chemical selector, two or more receptacles of floor treatment chemicals may be fluidly connected to a mixing tee. In operation, a user is capable of creating any number of cleaning solutions without the need for adding receptacles or switching chemical feed lines from one receptacle to another or without changing metering tips that are easily misplaced, incorrectly interconnected, or damaged. Thus, the treatment process is safer because there is less chemical handling. Similarly, use of a metering valve will allow the operator to create a very precise floor treatment solution.

It is preferred that one-way check valves be used throughout the apparatus. For instance, check valves may be included in: delivery lines that supply cleaning chemicals to the metering tee; lines that supply water to the metering tee; lines that supply cleaning solution to the pump; lines that supply cleaning solution to the spray gun; or in the metering tee, itself. The check valves prevent reversal of fluid and prevent contamination of one fluid with another.

Blower

The treatment apparatus also may include a modular blower assembly. The blower assembly may be hand-held and operate completely apart from the overall cleaning machine. The blower assembly may be used to dry areas physically separate from where the apparatus is stored. Because the blower assembly possibly is separate from the apparatus, it may also be used for other blowing functions, such as blowing leaves, grass, dirt or other debris. The blower assembly may be used with a detachable hand nozzle, a flexible nozzle, an extension wand, etc., thereby increasing the overall flexibility of the blower assembly. The blower assembly may utilize an integrated on/off switch and be powered by electricity supplied by any typical extension cord, including the power source of the apparatus. The blower may be configured to be stored on the apparatus in one of any number of convenient ways. It will be appreciated by one skilled in the art that having a modular blower assembly of this type is very beneficial to the overall functionality of a multifunctional floor treatment apparatus.

Storage

Another aspect of one embodiment of the present invention is that the chassis includes bins, trays, bays and other storage devices preferably within easy reach of the operator. The storage devices provide the operator with substantial flexibility when cleaning a large building or area that has many types of surfaces that may need treatment. Also, the apparatus provides for modular trash/supply bins that may be added to or removed from the apparatus quickly and easily so that the machine can be configured for one of any number of floor treatment activities.

In accordance with one embodiment of the present invention, an additional fluid tank retaining system is included on the chassis of the machine. The retention system allows virtually any hand pump sprayer or other container to be attached to the chassis for ease of transportation and operation.

Primary Pump

It is yet another aspect of the present invention to provide an apparatus equipped with a secondary fluid pump that supplies fluid to the main fluid pump prior to ignition. More specifically, one embodiment of the present invention includes a secondary, or priming pump, which is activated prior to the activation of the main fluid pump. Often it is desirable to introduce fluid into a main fluid pump prior to that pump's activation, thereby expelling trapped air that may cause damage to the main fluid pump motor from vapor lock or cavitation, for example. This priming process may be conducted manually, but that is time consuming, wherein the user manually adds fluid to the pump or bleeds the air therefrom. Alternatively, and preferably, one embodiment of the present invention is equipped with a secondary pump that is activated for a brief moment when the fluid discharge apparatus is initially activated, thus ensuring that the main fluid pump will be substantially free of trapped air upon activation.

Squeegee

It is another aspect of the present invention to provide a device that includes a squeegee adjacent to the floor treatment device, both generally in the middle of the machine. The squeegee effectively swings, or follows the path of the floor and does not rely on unvacuumized side squeegees to channel water to the main vacuumized squeegee. Thus, it offers as good or better fluid pick-up when the apparatus is turning than is capable with a walk behind scrubber, and far superior than typical small riders since it does not rely on smearing side squeegees. One embodiment of the present invention, employs a squeegee that pivots about the steering axis with a linkage that is supported by a roller and track mechanism. The absence of side squeegees mean less drag and better use of available energy. In addition, some embodiments of the present invention include an adjustable squeegee, a skirt or a shroud that minimally contacts the floor, thus reducing drag and sparing battery charge. Alternatively, some embodiments of the present invention include stops that contact the floor, without marring the same.

Use of the Device

Various aspects of the inventions discussed briefly above combine to provide an effective and efficient tool, useful in the treatment of numerous areas in and around commercial, industrial, institutional and public buildings. Moreover, due to the various aspects of the present invention, a sanitation maintenance worker may clean a particular room or facility more efficiently than previously possible. The present invention may be used in various cleaning operations such as burnishing, vacuuming, scrubbing, sanding, waxing, sweeping, sealing, painting, polishing, etc. In order to accomplish these tasks, the present invention may be equipped with various combinations of floor treatment devices. More specifically, one embodiment of the present invention is equipped with a plurality of brushes and squeegees to agitate and collect debris from a flooring surface. In addition, suction mechanisms may be employed such that fluids and/or dry particulate matter are transferred into a container. It is also envisioned that one embodiment of the present invention include at least one solution applicator positioned adjacent to the scrub brushes, wherein solution is injected onto the surface after, or prior to, agitation by the brushes. The debris-entrained solution is then collected by the squeegee and subsequently vacuumed into the holding tank or expelled out of the chassis to an outside reservoir. The brushes and/or solution used in this embodiment may be adapted to clean, sweep, paint, burnish, sand, strip, varnish or wax a floor. It will be appreciated by one skilled in the art that any type of solution adapted to treat any flooring surface may be employed without departing from the scope of the present invention.

It is yet another aspect of the present invention provide a floor treatment apparatus that can be used in various floor maintenance operations. More specifically, one embodiment of the present invention is adapted for interconnection to a plurality of devices to perform a variety of floor treatment operations. It is envisioned that one embodiment of the present invention be capable of quick removal of certain treatment devices such that different devices may be then added to quickly change the scope of the apparatus, thereby providing a device adapted to scrub, clean carpets, wax floors, burnish floors, remove wax or varnish from floors, vacuum, etc. Thus, it is contemplated, that this system may be used for a plurality of cleaning or floor treatment operations.

Brush Drive

Another aspect of the present invention is to provide a single motor using a two sided belt to drive at least two brushes used as floor treatment devices in accordance with embodiments of the present invention. In one embodiment, the motor is operably connected to a first and second brush that are used to engage the floor surface. The brushes can be adapted for use on carpeted surfaces or hard floor surfaces. The first and second brush rotate in opposite directions such that debris is lifted between the brushes. For example, the first brush may be rotating in a clockwise direction, whereas the second brush is rotating in a counter-clockwise direction. The two sided belt used to interconnect the motor to the first and second brushes may also be connected to a constant force tension member. The constant force tension member is spring loaded on the “slack” side of the belt. Thus, providing a repeatable belt tension regardless of part or assembly tolerances.

In one embodiment, the tension member is an idler pulley with a spring and ratchet mechanism attached thereto. The belt is maintained at a relatively constant tension due to the spring loaded self tensioning member. In accordance with one embodiment, the motor pulley and brush pulleys are set at fixed locations and the tensioning member is adjustable. This particular configuration eliminates the need for an operator to manually set the tension on the belt.

In accordance with one embodiment of the invention, a wide area vacuum attachment may be selectively incorporated into the cleaning device as one cleaning mechanism. The wide area vacuum may incorporate dual brushes and may be driven by one motor as described above. Alternatively, the dual brushes may be driven by a pair of motors if desired. The wide area vacuum brushes may be about 16″ to about 30″ in length, and more preferably from about 24″ to about 28″, with a most preferred length of about 26″. Other preferable lengths of brushes include lengths of about 16″, 20″, or 24″. However, as one skilled in the art would recognize, the brushes could be virtually any desired length, width, and configuration.

Brush Mounting

A further aspect of the present invention is to provide a mounting system for the non-drive side of a cylindrical brush. The cylindrical brush may be used on either hard floor surfaces or carpeted surfaces. Generally, cylindrical brushes collect unwanted debris (i.e., hair, dirt, etc.) around their bearing housing and spindle area. In one embodiment, the mounting system comprises a brush hub cap that substantially prevents infiltration of debris into the bearing housing and spindle area.

The brush hub cap on the non-drive end includes an end cap, bearings, an end cap, a brush/bearing insert, and a spring clip. The insert protects the bearing housing from debris agitated by the brush by creating a longer path of travel for the debris to get to the bearing housing and spindle.

The brush may be held in place by a spring clip that is attached to the brush housing. The operator of the cleaning device or maintenance personnel may be able to access the brush on the non-drive side without having to remove any parts. The spring clips may provide for easy insertion and removal of a brush without requiring any substantial disassembly of the housing or bearings

The brush mounting on the drive end includes a drive hub, a pulley, a drive shaft, bearings, and a drive housing. A tight fight between the drive shaft and drive housing helps protect the bearing housing from debris agitated by the brush.

Spray Nozzle

A still further aspect of the present invention is to furnish an apparatus that mixes and a base solution, for instance water, and a cleaning solution at the point of application. Specifically, a nozzle design is provided that functionally uses a siphon created by water pressure to mix the water and a cleaning solution outside of the nozzle. In one embodiment, the valve design uses an orifice sealed by the pressurized water flow to create a syphon to mix the water and cleaning solution at the point of contact. The mixed solution continues out of the nozzle mixing point to strike a dispersion surface that sprays the mixed solution in a relatively wide angle flat spray pattern. The base solution is injected into the nozzle at a pressure, whereas the cleaning solution is present in its respective line under no pressure. The flow of the cleaning solution is prompted by the passing of the base solution across the opening of the chemical solution line.

In one embodiment of the present invention, a plurality of nozzles can be combined to form a cleaning solution mixing block. The mixing block directs pressurized water to one or more of a plurality of orifices or nozzles where various different chemicals can be mixed. The orifice size of each cleaning solution line can be the same thereby allowing each cleaning solution to be mixed with the base solution at the same ratio. Alternatively, the orifice size of each cleaning solution line may vary to create different ratios of cleaning solution to base solution. As stated above, one cleaning solution may be mixed with the base solution at a time. This may be preferable if the user wishes to change from deep cleaning to light cleaning and/or interim cleaning. However, several cleaning solutions can be mixed at the same time by passing pressurized water over a number of cleaning solution orifices. Cleaning solutions can be selected by use of a selector attached to the mixing block directly or at the user interface of the cleaning device. The selector may be a mechanical or electronic actuator that controls a valve that directs water to the one or more nozzles.

Remote Control

It is yet another aspect of the present invention to provide a highly mobile floor treatment apparatus that can include a car washer assembly. As will be appreciated by those skilled in the art, if so configured, the device could include a car washer wand connected to appropriate pumps and could be utilized to pre-clean heavily soiled areas prior to final cleaning with use of the device.

It is still another aspect of the present invention to provide a floor treatment apparatus that does not require direct contact with an operator to perform its tasks. More specifically, one embodiment of the present invention is adapted to be remote controlled. This embodiment of the present invention is equipped with remote control mechanisms and software currently known in the art, such as taught by U.S. Pat. No. 6,625,843 to Kim et al., which is incorporated in its entirety herein. In addition, this embodiment of the present invention may be equipped with the plurality of cameras such that offsite monitoring and control may be performed. In a related embodiment of the present invention, software is installed in the cleaning apparatus such that human contact or monitoring is not required. More specifically, one embodiment of the present invention is adapted to learn its environment as it operates in an area such that remote controlling is not required. Alternatively, it is well within the scope of this invention to preprogram the dimension of floored surfaces into the smart treatment device, wherein the device is parameterized with the surface dimensions before the task is initiated. Apparatus of this type are known in the art, such as the Roomba™ device by iRobot Corporation, aspects of which are described in U.S. Pat. Nos. 6,594,844 and 6,535,793, which are both incorporated in their entirety herein.

Safety

It is another aspect of the present invention to provide a cleaning apparatus that is safe and comfortable to use. More specifically, one embodiment of the present invention includes an operator platform. This platform allows the operator to stand on the device during the treatment operation, thus increasing productivity and lowering the chances of injury or fatigue to the operator. It another embodiment of the present invention, a seat is provided wherein the operator may comfortably sit while completing his or her task. Other safety and comfort features such as rails, pads, and belts, may be provided depending on the needs of the operator.

Floating Deck

It is still another aspect of the present invention to provide a counter balance mechanism that enables the deck used in accordance with embodiments of the present invention to float on an uneven floor/surface with a substantially consistent downward force. In one embodiment, a deck is attached to the chassis of the present invention through a counter balance mechanism that utilizes a unique geometry along with springs to enable the deck to continually adjust itself, thereby obviating the need for an electronic controller or continual manual adjustment of the deck by the operator. The use of a floating deck in accordance with embodiments of the present invention acts to provide a substantially consistent counter balance force over a range of movement of the cleaning device, unlike previous prior art.

A spring is used in accordance with one embodiment of the present invention to ensure that no excessive forces are applied to the floor/surface due to the weight of the deck and its attachments. As the topography of the floor changes, the force applied to the counter balance mechanism by the spring is adjusted because the lever arm of the spring relative to the counter balance mechanism changes. At substantially the same time, the lever arm of the force applied by the deck to the counter balance mechanism changes opposite to the spring lever arm. For example, when the floor contour changes in one direction, the lever arm of the spring may increase while the lever arm applied by the deck's weight may decrease. The relative adjustments of the lever arms in accordance with embodiments of the present invention act to provide and/or maintain contact between the deck and the floor/surface through a broad range of movement.

The Summary of the Invention is neither intended nor should it be construed as being representative of the full extent and scope of the present invention. Some aspects of the present invention are set forth in various levels of detail in the Summary of the Invention, as well as in the attached drawings and the Detailed Description of the Invention. No limitation as to the scope of the present invention is intended by either the inclusion or non-inclusion of elements, components, etc. in this Summary of the Invention.

### BRIEF DESCRIPTION OF THE DRAWINGS

The accompanying drawings, which are incorporated in and constitute a part of the specification, illustrate embodiments of the invention and together with the general description of the invention given above and the detailed description of the drawings given below, serve to explain the principles of these embodiments.

FIG. 1 is a perspective view of one embodiment of the present invention showing an operator standing on the platform thereon;

FIG. 2 is a perspective of an alternate embodiment of the present invention that is configured for fluid extraction, and which is controlled by at least one joy stick;

FIG. 3 is a perspective view and alternative embodiment of the present invention that is configured for burnishing operations;

FIG. 4 is a perspective view of an alternative embodiment of the present invention that is equipped with moveable brushes that are adapted to swing out to more efficiently treat a floor surface, and which also includes a wand for selectively cleaning difficult to reach areas;

FIG. 5 is a perspective view of an alternative embodiment of the present invention that is designed to rotate about an 360° axis without significantly traversing in other directions;

FIG. 6 is a perspective view of an alternative embodiment of the present invention that is designed to reach tight areas of floor surface;

FIG. 7 is a detailed perspective view of the embodiment shown in FIG. 6, showing the steering wheel, brush, and squeegee assembly used therewith;

FIG. 8 is a top plan view of a flooring surface;

FIG. 9 are bottom plan views showing configurations of steering, cleaning, and power mechanisms;

FIG. 10 is a bottom plan view of an alternate embodiment of the present invention showing an alternate configuration of steering, cleaning, and power mechanisms;

FIG. 11 is a perspective view of an alternative embodiment of the present invention that is adapted to be remotely controlled;

FIG. 12 are views of a rotatable squeegee for use in one embodiment of the present invention;

FIG. 13 are views of a waste fluid system showing a strain basket and a drainage port of one embodiment of the present invention;

FIG. 14 are views of the rear cowling and battery tray of one embodiment of the present invention;

FIG. 15 is a perspective view of a control panel and handles of one embodiment of the present invention;

FIG. 16 is a perspective view of an operator platform with a plurality of switches of one embodiment of the present invention;

FIG. 17 is are views of a seat of one embodiment of the present invention;

FIG. 18 are views of a tank and front cowling of one embodiment of the present invention;

FIG. 19 are views of a vacuum fan interconnected to the front cowling of one embodiment of the present invention;

FIG. 20 is a right elevation view of one embodiment of the present invention showing the waste water return hose;

FIG. 21 is a perspective view of a base solution tank and fluid recovery tank in accordance with embodiments of the present invention;

FIG. 22 is a schematic of a single motor drive assembly for cylindrical brushes in accordance with one embodiment of the present invention;

FIG. 23 shows a perspective view of a wide area vacuum attachment in accordance with another embodiment of the present invention;

FIG. 24a shows a perspective view of a bearing protection system for the non-drive side of a rotating cylindrical brush in accordance with yet another embodiment of the present invention;

FIG. 24b shows a cross sectional view of a bearing protection system for the non-drive side of a rotating cylindrical brush in accordance with embodiments of the present invention;

FIG. 24c shows a cross sectional view of a bearing protection system for the non-drive side of a rotating cylindrical brush in accordance with embodiments of the present invention;

FIG. 24d shows a perspective view of a non-drive side of a brush housing that provides external access to a brush in accordance with embodiments of the present invention;

FIG. 25a shows a perspective view of a bearing protection system for the drive side of a rotating cylindrical brush in accordance with embodiments of the present invention;

FIG. 25b shows a cross sectional view of a bearing protection system for the drive side of a rotating cylindrical brush in accordance with embodiments of the present invention;

FIG. 25c shows a cross sectional view of a bearing protection system for the drive side of a rotating cylindrical brush in accordance with embodiments of the present invention;

FIG. 26 shows a side cross-sectional view of a spray nozzle in accordance with a further embodiment of the present invention;

FIG. 27 shows an isometric view of the spray nozzle in accordance with yet a further embodiment of the present invention;

FIG. 28 shows a side cross-sectional view of a collection of spray nozzles creating a mixing block in accordance with still a further embodiment of the present invention;

FIG. 29 shows a front cross-sectional view of a collection of spray nozzles creating a mixing block in accordance with one embodiment of the present invention;

FIG. 30 shows an isometric view of the mixing block in accordance with embodiments of the present invention;

FIG. 31 shows an isometric view of the counter balance mechanism in accordance with one embodiment of the present invention;

FIG. 32A is a first representative diagram depicting a counter balance mechanism in accordance with embodiments of the present invention;

FIG. 32B is a second representative diagram depicting a counter balance mechanism in accordance with embodiments of the present invention;

FIG. 33A shows a side view of the counter balance mechanism attached to a chassis of a cleaning device used to hold a cleaning deck in accordance with embodiments of the present invention;

FIG. 33B shows a side view of the counter balance mechanism attached to a chassis of a cleaning device used to hold a cleaning deck in an alternative configuration; and

FIG. 34 shows an additional tank retention member for use with the cleaning device in accordance with embodiments of the present invention.

To assist in the understanding of the present invention the following list of components and associated numbering found in the drawings is provided herein:

| Component | # |
|---|---|
| Floor treating apparatus | 2 |
| Platform | 4 |
| Operator | 6 |
| Chassis | 8 |
| Bottom surface of chassis | 10 |
| Brush | 12 |
| Rotating brush | 13 |
| Scrubber | 14 |
| Squeegee | 16 |
| Wheel | 18 |
| Steering wheel | 20 |
| Joy stick | 22 |
| Handle Grip | 24 |
| Powered wheel | 26 |
| Burnishing pad | 28 |
| Swinging brush | 30 |
| Wand | 32 |
| Hose | 34 |
| Swing arm | 36 |
| Bearing | 38 |
| Track | 40 |
| Pivot point | 42 |
| Handle | 44 |
| Cam | 46 |
| Strainer basket | 48 |
| Waste tank cover | 49 |
| Waste fluid intake | 50 |
| Main Storage Tank | 51 |
| Clean fluid intake | 52 |
| Fitting | 54 |
| Flange | 56 |
| Waste fluid bag | 58 |
| Mandrill | 60 |
| Drain hose | 62 |
| Band Clamp | 64 |
| Rear cowling | 66 |
| Battery | 68 |
| Tray | 70 |
| Drink holder | 72 |
| Cowling pad | 74 |
| Control panel | 76 |
| Fastener | 77 |
| Operator presence switch | 80 |
| Throttle | 82 |
| Seat | 84 |
| Adjustment Mechanism | 85 |
| Hook | 86 |
| Front cowling | 88 |
| Light | 89 |
| Vacuum fan | 92 |
| Vacuum exhaust channels | 94 |
| Waste H2O return hose | 96 |
| Hose channel | 98 |
| Tip over stops | 100 |
| Base Solution Tank | 102 |
| Fluid Recovery Tank | 104 |
| Base Solution Tank Orifice | 106 |
| Motor Pulley | 108 |
| First Brush Pulley | 110 |
| Second Brush Pulley | 112 |
| Third Pulley | 113 |
| Timing Belt | 114 |
| Tensioning Member | 115 |
| Idler Pulley | 116 |
| Spring and Ratchet Member | 118 |
| Wide Area Vacuum | 120 |
| Brush | 121 |
| Bearing Insert | 122 |
| Bearing | 124 |
| Shaft | 126 |
| End Cap | 128 |
| Non-Drive Side Flange | 129 |
| Spring Clip | 130 |
| Housing | 131 |
| Drive Shaft | 132 |
| Drive Side Flange | 133 |
| Brush Pulley | 134 |
| Bearing | 136 |
| Drive Housing | 138 |
| Drive Pin | 140 |
| Drive Hub | 141 |
| Spray Nozzle | 142 |
| Base Fluid Flow | 144 |
| Cleaning Solution Flow | 146 |
| Base Solution Orifice | 148 |
| Siphon Point | 150 |
| Dispersion Surface | 152 |
| Output Spray | 154 |
| Attachment Point | 156 |
| Mixing Block | 158 |
| Valve Housing | 159 |
| Base Fluid Flow | 160 |
| Base Fluid Opening | 162 |
| Cleaning Solution Flow | 164 |
| Cleaning Solution Opening | 166 |
| Base Fluid Exit Orifice | 168 |
| Siphon Point | 170 |
| Dispersion Surface | 172 |
| Output Spray | 174 |
| Selector | 176 |
| Selection Valve | 178 |
| Nozzle | 180 |
| Counter Balance Mechanism | 182 |
| Base Plate | 184 |
| Side Plate | 186 |
| Balance Spring | 188 |
| Interconnection Member | 190 |
| Pivot Bolt | 192 |
| Deck Attachment Point | 194 |
| Spring Attachment Point | 196 |
| Spring Adjustment Member | 198 |
| Pivot Point | 200 |
| Linking Member | 201 |
| Cleaning Deck | 202 |
| Attachment Member | 204 |
| Retention Member | 206 |
| Body | 208 |
| Connection Arm | 210 |

It should be understood that the drawings are not necessarily to scale. In certain instances, details which are not necessary for an understanding of the invention or which render other details difficult to perceive may have been omitted. It should be understood, of course, that the invention is not necessarily limited to the particular embodiments illustrated herein.

### DETAILED DESCRIPTION

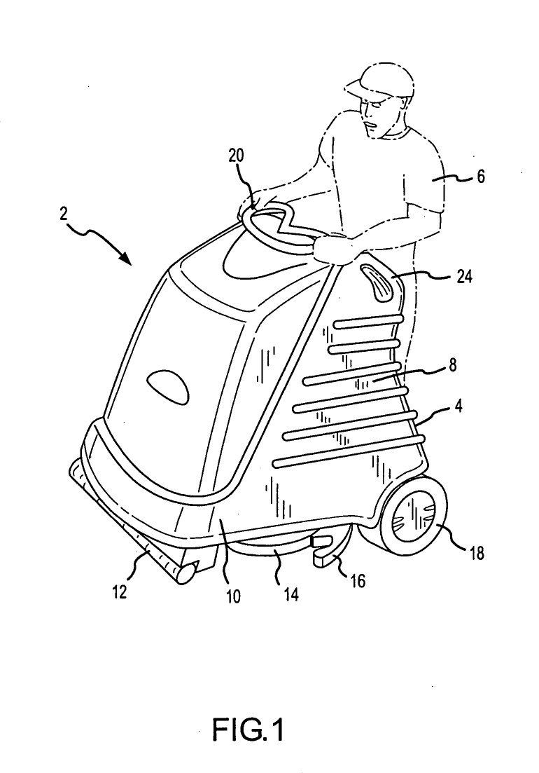

Referring now to FIGS. 1-34 an apparatus 2 for cleaning or otherwise treating a floor surface is shown. More specifically, one embodiment of the present invention includes a chassis 8 with a platform 4 that is adapted to support the weight of an operator 6, thus increasing the efficiency of the entire floor treatment operation. In addition, various cleaning or floor treatment components may be interconnected to the bottom surface 10 of the chassis, such as brushes 12, scrubbers 14, squeegees 16, vacuum shoes, etc.

The chassis 8 also includes a plurality of wheels 18 operably interconnected to the bottom surface 10 to enable steering and provide stability. It is contemplated that the operator 6 will stand on the platform 4 and steer the apparatus 2 with either a steering wheel 20 or other type of steering mechanism, such as a joy stick 22. Such an embodiment of the present invention enables the floor surface to be cleaned or otherwise treated more efficiently, since the operator 6 does not have to push or pull an often heavy apparatus 2. In addition, since the human component of powering or otherwise moving the apparatus 2 is omitted, more consistent flooring treatment is achieved, thereby saving materials and reducing costs of the entire operation.

Referring now to FIG. 1, one embodiment of the present invention is shown. More specifically, a chassis 8 which includes the platform 4 adapted to support an operator 6 during the floor treatment operation is shown. The operator 6 preferably stands on the platform 4 that is generally parallel to the flooring surface. Preferably, the platform 4 is tilted, rear edge higher than the front edge, between about 3 to 8 degrees to increase ergonomics. However, as it will be appreciated by one skilled in the art, other support devices, such as seats, which may be operably folded into the chassis 8, may be provided to increase the comfort level of the operator 6. In addition, the embodiment of the present invention shown in FIG. 1 is equipped with a steering mechanism, such as a wheel 18, that allows the operator 6 to easily maneuver the apparatus 2 around the flooring surface.

The chassis 8 is constructed of any material, but preferably hard plastic will be used to reduce the weight of the apparatus 2. As shown herein, a plurality of wheels 18 are operably interconnected to the rear of the apparatus 2 to provide stability and perhaps power for locomotion. In addition, a squeegee 16 is included that is adapted to extract or funnel water or debris to a location where it is extracted via vacuum into a container generally, but not always, located at least partially inside the chassis 8. Further, this embodiment of the present invention includes a brush 12 that is used to agitate the flooring surface to loosen dirt, wherein spray nozzles may be employed situated behind the brush 12 to treat the flooring and capture the dirt so that it can be gathered by the squeegee 16 and suction system of the apparatus 2.

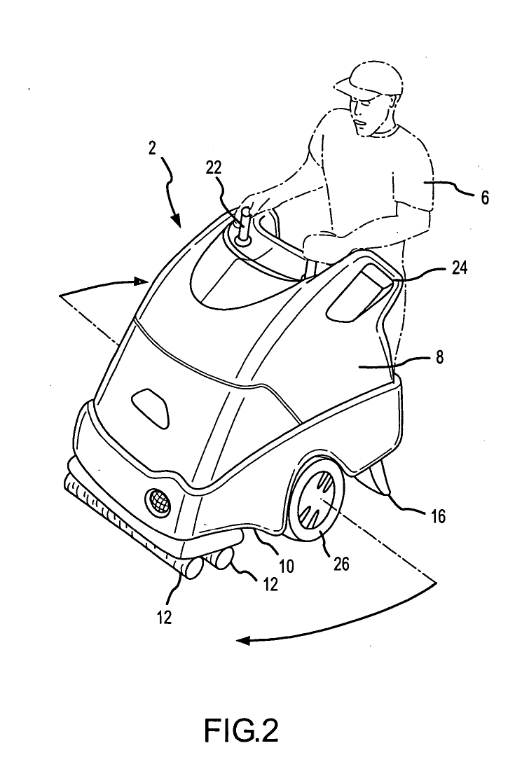

Referring now to FIG. 2, an alternate embodiment of the present invention that is used mainly for fluid extraction is shown. This embodiment of the present invention is similar to the apparatus described above, however alternate components are interconnected to the bottom surface 10 of the chassis 8 such that the apparatus is adapted to efficiently capture fluids or debris deposited on a floored surface. More specifically, this embodiment of the present invention is equipped with at least one brush 12 adapted to agitate water and/or debris and a squeegee 16 that is positioned adjacent to the rear surface of the chassis 8 that contains fluid and debris as the apparatus 2 moves forward. In one embodiment of the present invention, a suction device, such as a vacuum shoe, is positioned near the squeegee 16 such that dirty water is vacuumed from the surface and transferred back into a tank situated inside or adjacent to the chassis 8. Alternatively, another embodiment of the present invention is provided with a squeegee 16 with a plurality of suction holes that are the terminus of conduits that transport waste water to the storage tank.

In the illustrated embodiment, the operator 6 is able to control the apparatus 2 with a plurality of joy sticks 22. In addition, hand grips 24 are provided on the sides of the operator 6 to increase safety. Further, this embodiment of the present invention employs powered wheels 26 that allow the entire system to rotate on a single vertical axis without substantially transitioning in other directions. More specifically, this embodiment of the present invention is capable of performing a 360° turn, which aids cleaning of tight spaces.

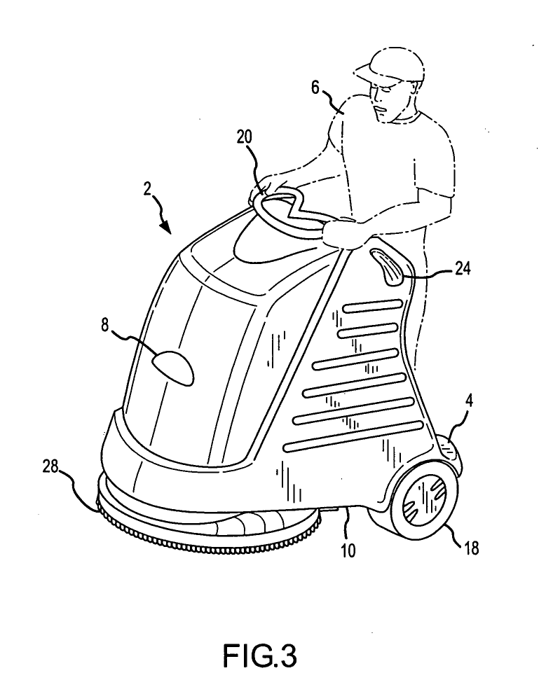

An alternate embodiment of the present invention that is used for burnishing is shown in FIG. 3. This embodiment of the present invention includes a burnishing pad 28 operably interconnected to the bottom surface of the chassis 10. As before, the operator 6 stands on a platform 4 built into the chassis 8. One skilled in the art will appreciate that this embodiment of the present invention may also include a device for suctioning debris left over from the burnishing process, such as dust or wax particulates, for example.

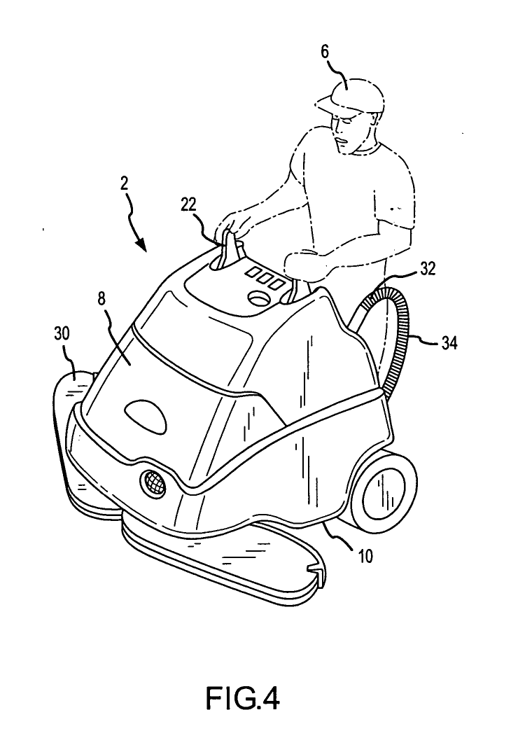

Referring now to FIG. 4, an alternate embodiment of the present invention that employs swinging brushes 30 is shown. This embodiment of the present invention is very similar to those described above, however the brushes 30 used to agitate, scrub, or burnish are rotatably interconnected to the bottom surface 10 of the chassis 8. More specifically, the brushes 30 of this embodiment are capable of independently folding inwardly, thereby efficiently cleaning the interior portion of a floor when the apparatus is operating near a vertical surface such as a wall. As shown herein, the brushes 30 are independently movable and preferably spring loaded outward such that contact with a vertical surface causes the brush 30 to fold under the chassis 8. Alternatively, as one in the art will appreciate, the orientation of the brushes may be controlled by the operator. In addition, a wand 32 interconnected to a hose 34 may also be employed with this embodiment of the present invention to allow for selective application of cleaning solution or suction.

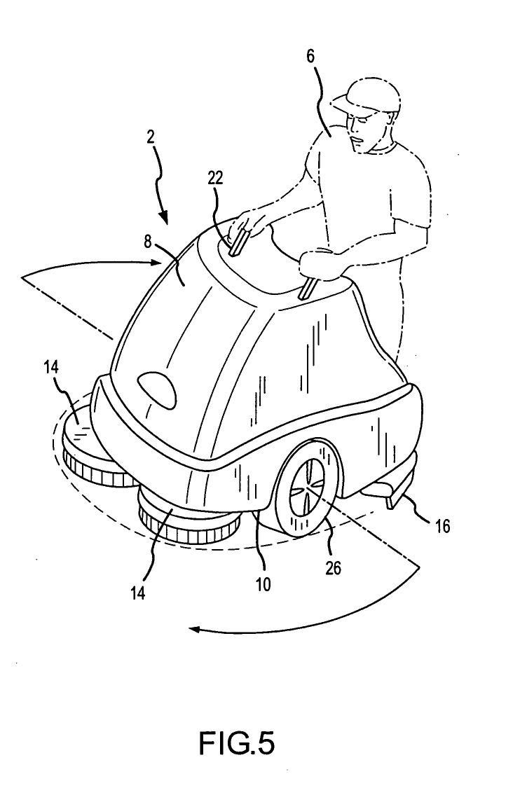

Referring now to FIG. 5, another embodiment of the present invention that utilizes centered powered wheels 26 is shown. More specifically, this embodiment of the invention is similar to those described above, however it is equipped with a plurality of wheels 26 that allow a 360° turning capability. This embodiment of the present invention is also similarly adapted for cleaning the surface of a floor with a brush 12 or a plurality thereof that is used to agitate the dirt wherein a squeegee contains and suctions debris into a container.

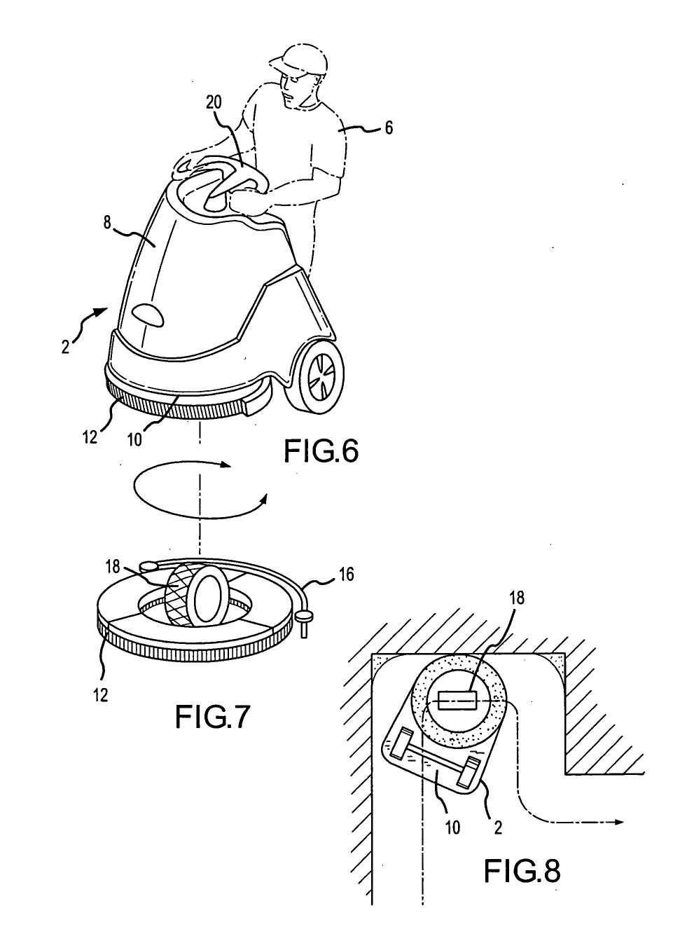

Referring now to FIGS. 6-8, an alternate embodiment of the present invention is shown that is equipped with a wheel 18 with brushes 12 there around for cleaning in all directions. This embodiment of the present invention is equipped with brushes 12 that allow for cleaning or agitation of the flooring surface in any direction the apparatus 2 is moving, thus efficiently cleaning flooring without having to make multiple passes over the surface.

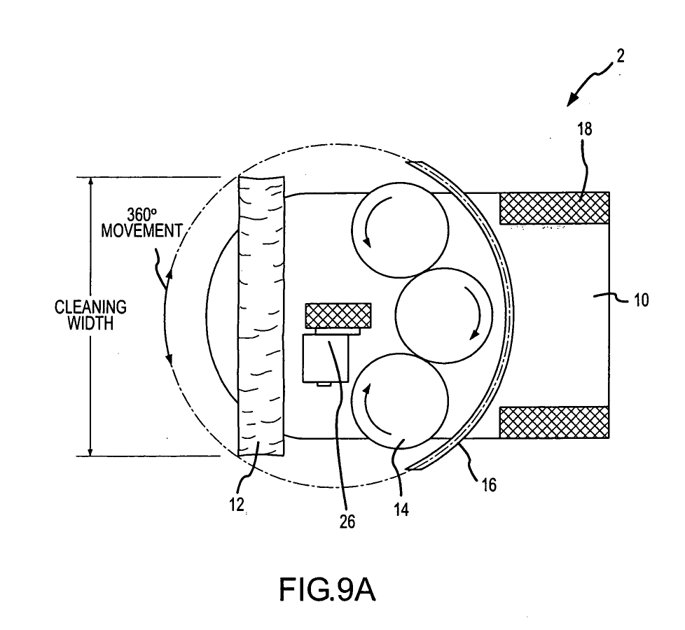

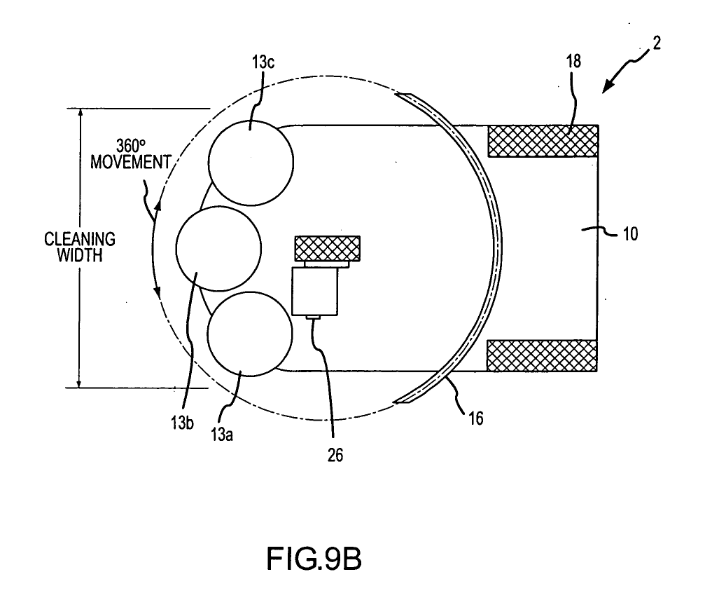

Referring now to FIG. 9, one configuration of cleaning components interconnected to the bottom surface 10 of the chassis 8 is shown. More specifically, one embodiment of the present invention is adapted to either sweep or clean a floor. In the illustrated embodiment, a presweeping brush 12 agitates the carpet or hardwood floor to loosen debris. Next, rotating scrubbing brushes further agitate the surface and perhaps add fluid and cleaning solution thereto to help loosen and contain any loose debris. Finally, a squeegee 16 and preferably a suction system is provided that captures the dirty water and as the apparatus is moved forward. As shown herein, the drive unit is the center wheel 26, which is also adapted to selectively rotate upon steering commands from the operator 6.

FIG. 9B shows a configuration of cleaning components interconnected to the bottom surface 10 of the chassis 8 similar to what was shown in FIG. 9A. The difference, however, is that the pre-sweeping brush 12 has been replaced by three scrub brushes or three rotating brushes, 13A, 13B and 13C that may be used to either sweep, burnish or combinations thereof a floor surface. The brushes can rotate at speeds desired by the operator or at preselected speeds and in directions selected by the operator or in pre-selected directions.

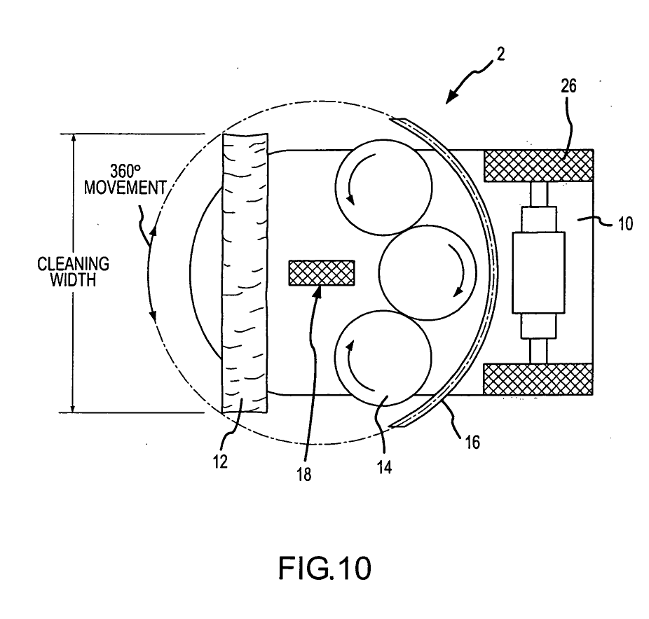

Referring now to FIG. 10, an alternate configuration of the cleaning components interconnected to the bottom surface 10 of the chassis 8 is shown. More specifically, this configuration is substantially similar to that shown above in FIG. 9, however, the drive mechanism of the apparatus is a transaxled power plant that provides power to the rear wheels 26, wherein the steering is performed by a front wheel.

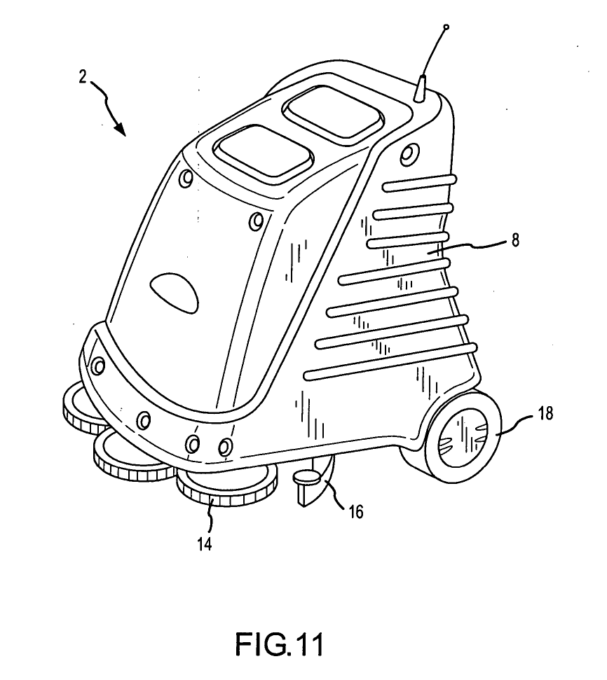

Referring now to FIG. 11, yet another embodiment of the present invention performs a floor treatment operation without the need of physical human contact is shown. More specifically, this embodiment of the present invention is remote controlled or otherwise intelligent such that it cleans a floored surface without the direct contact of an operator. This embodiment of the present invention may be configured for any task, such as scrubbing, sweeping, vacuuming, burnishing, carpet cleaning, waxing, surfacing, cleaning, etc. It is envisioned that the operator be in a separate location, perhaps offsite from the actual cleaning operation, and aided by remote viewing devices. Alternatively, one embodiment of the present invention is programmed with the ability to automatically treat a floor surface, wherein the dimensions of the surface are either programmed into or learned as the apparatus is in use, thereby alleviating any need for human contact with the apparatus. This embodiment of the present invention may be deployed from a storage location automatically wherein quick disconnects to fluid sources or waste receptacles are remotely joined to it such that filling and emptying tanks or waste containers inside the chassis 8 is done without the need of a human operator as well. This embodiment of the present invention may be used in areas where it is dangerous for humans to operate, such as nuclear power plants, areas where asbestos exposure is likely, etc.

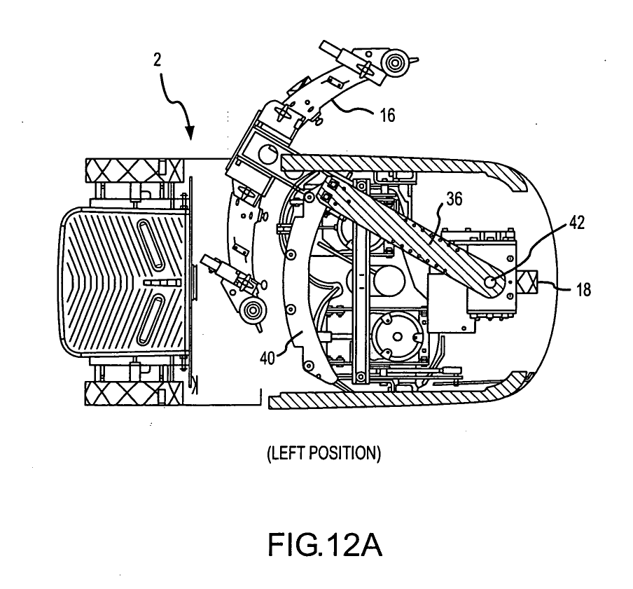

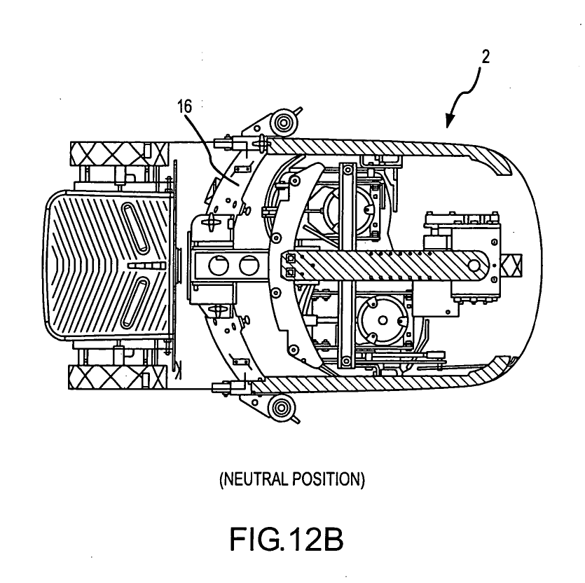

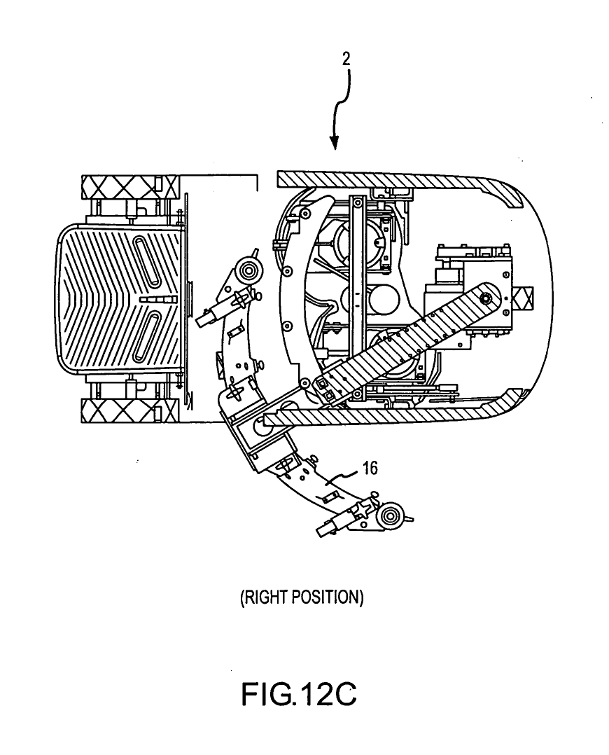

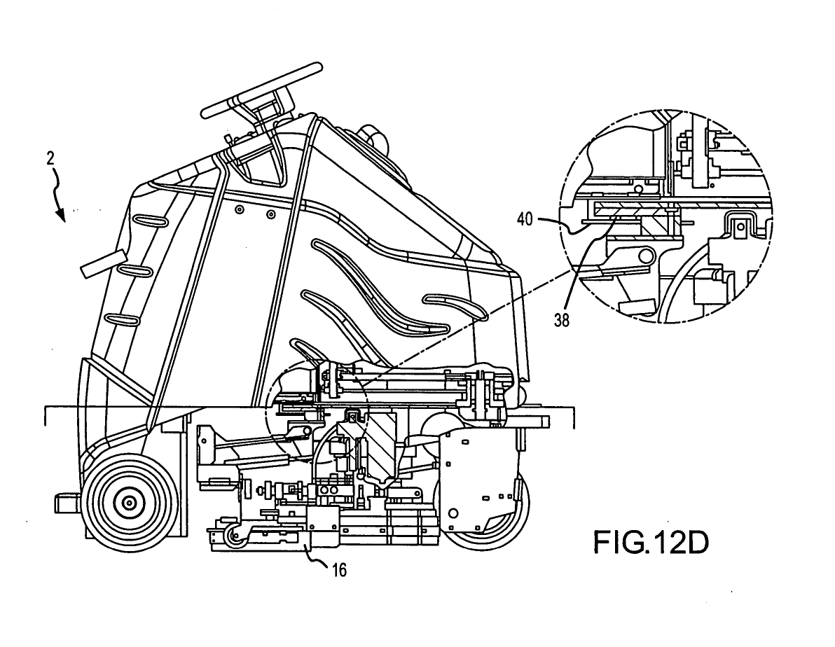

Referring now to FIG. 12, a squeegee 16 for use in one embodiments of the present invention is shown. More specifically, some embodiments of the present invention include a pivot mechanism that allows the squeegee 16 to remain in place when the floor treating apparatus 2 is turning. Thus, the amount of fluid extracted when the apparatus 2 is making a tight turn is increased. In the illustrated embodiment, the squeegee 16 is connected to a swing arm 36 that pivots about a point adjacent to the front wheel 18 of the apparatus. The swing arm 36 is supported via rollers or bearings 38 on a track 40 that maintain the squeegee's 16 vertical position relative to the floor. Upon making a right or left hand turn, friction will tend to keep the squeegee 16 in a straight line, following the original path of the vehicle. Once a new line of travel is established, the squeegee 16 will fall back in place substantially under the apparatus 2. FIG. 12a shows the squeegee 16 in its upmost left position, while FIG. 12c shows the squeegee in its upmost right position. FIG. 12b shows the squeegee in a neutral position while FIG. 12d shows the squeegee in a neutral position but from a side view.

The squeegee 16 of one embodiment of the present invention is provided with a plurality of wheels that interface with the floor to maintain the vertical clearance of the squeegee assembly. In addition, side rollers may be provided that prevent the squeegee 16 from contacting a vertical surface, such as a wall. These wheels and various portions of the squeegee assembly may be selectively adjustable such that the width of the squeegee 16 and the placement of the wheels (squeegee height) may be altered at will.

As shown herein, the swing arm 36 connects to a pivot 42 that utilizes the momentum of the squeegee 16 to swing it from the apparatus 2. However, one skilled in the art will appreciate other methods of transitioning the squeegee 16 from the floor treatment apparatus 2 may be utilized without departing from the scope of the invention. More specifically, a motorized system may be employed that is in communication with the steering system of the vehicle such that rotation of the steering wheel will swing the squeegee 16 away from the apparatus 2 in a predetermined manner.

An actuation system that selectively raises the squeegee 16 from the floor may also be included as shown in FIG. 12E. In accordance with some embodiments of the present invention, a handle actuated leverage system 44 is used and is in mechanical communication with a cam 46. The cam allows the user to apply minimal force to the handle 44 adjacent to the control panel to raise and lower the squeegee 16. One skilled in the art will also appreciate that this function may be performed alternatively with a motor.

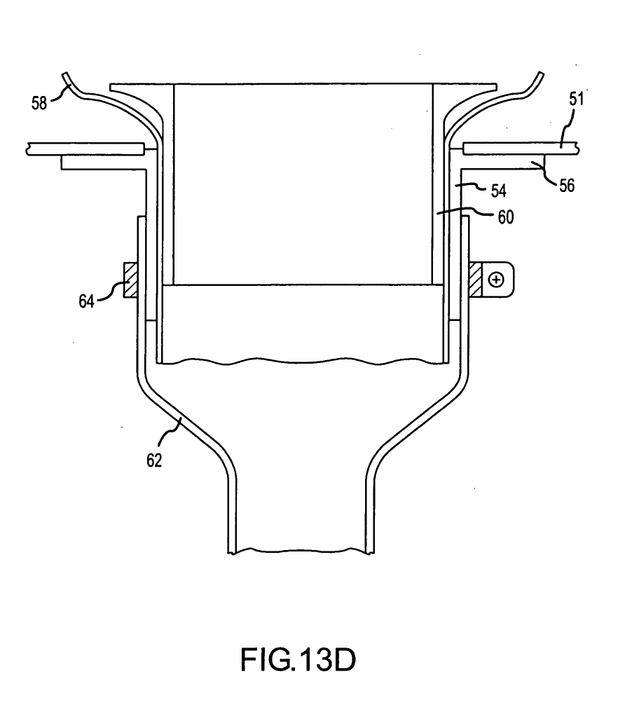

Referring now to FIG. 13, a recovery tank strainer basket 48 of one embodiment of the present invention is shown. Recovery tanks of some embodiments of the present invention are constructed out of resiliently deflectable material, such as a plastic bag. The bag is inserted into the clean fluid tank 51 of the apparatus. Once the clean fluid is transferred to the floor treatment tool of the apparatus, waste water may be suctioned into the waste fluid tank, thus expanding the bag and occupying the space once occupied by now dispensed clean fluid. Often, small metal shavings, wood splinters, glass, etc., may be suctioned with the waste fluid and deposited into the waste fluid tank, which may produce rips or tears in the bag and ultimately lead to leakage and contamination of the cleaning fluid. Thus, it is desirable to have a system that captures any dangerous debris such that it does not come in contact with the waste fluid tank. One embodiment of the present invention thus includes a strain basket 48 connected to the cover 49 of the waste fluid tank. In the illustrated embodiment, a generally rectangular straining device constructed of a rigid material with a plurality of apertures there through is provided. As the waste water is deposited into the tank through the cover, any large debris is captured by the strain basket 48. One skilled in the art will appreciate that any sized aperture may be employed to dictate the size of debris that is captured. Also, it should be specifically understood that any shape of strain basket 48 may be used without departing from the scope of the invention.

Referring now specifically to FIG. 13D, a fluid discharge system that is connected to the waste water tank 58 of one embodiment of the present invention is shown. More specifically, a fitting 54 with a flange 56 may be used that is connected to the main storage tank 51 of the apparatus. Preferably, the fitting 54 is spun at a high rate of speed and engaged with an aperture in the tank 51, thus creating friction induced heat between the two surfaces and welding them together. The opening of the waste water bag 58 is then fed through the fitting 54 and a mandrill 60 is added to sandwich the waste water bag 58 there between. The mandrill 60 is made out of a rigid material, such as aluminum to ensure an open flow path. A drain hose 62 is slid over the outer surface of the fitting 54 and is secured with a clamp 64. One skilled in the art will appreciate that the drain hose 64 is generally capped during use, wherein the user disconnects the cap to drain the waste water from the bag 58. To ensure that the bag 58 is entirely empty, a new solution may be added to the tank, thus squeezing the bag 58 to expel all the waste water contained therein.

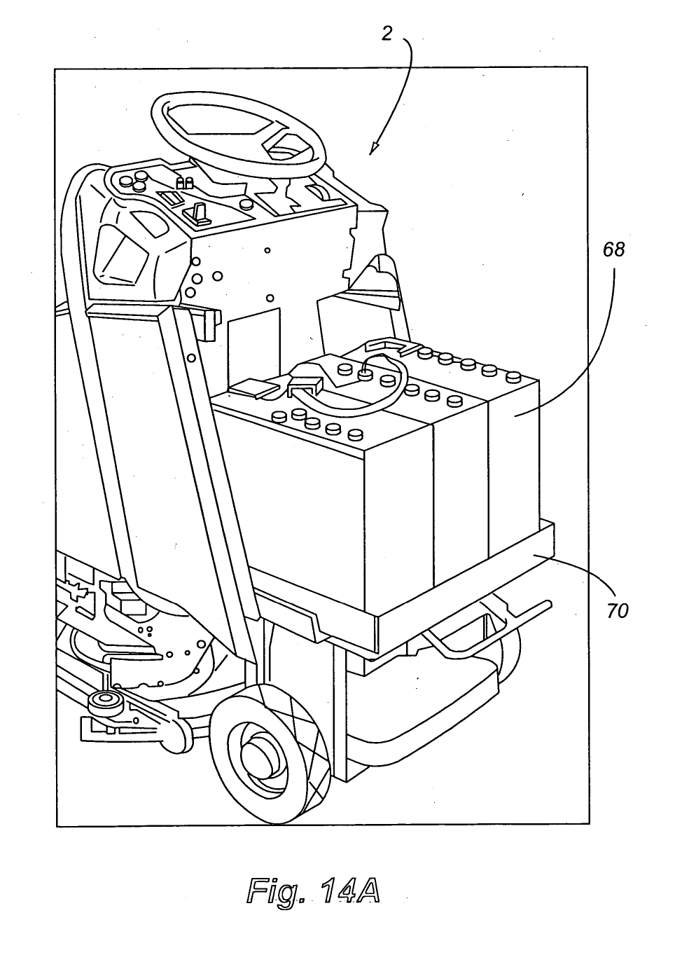

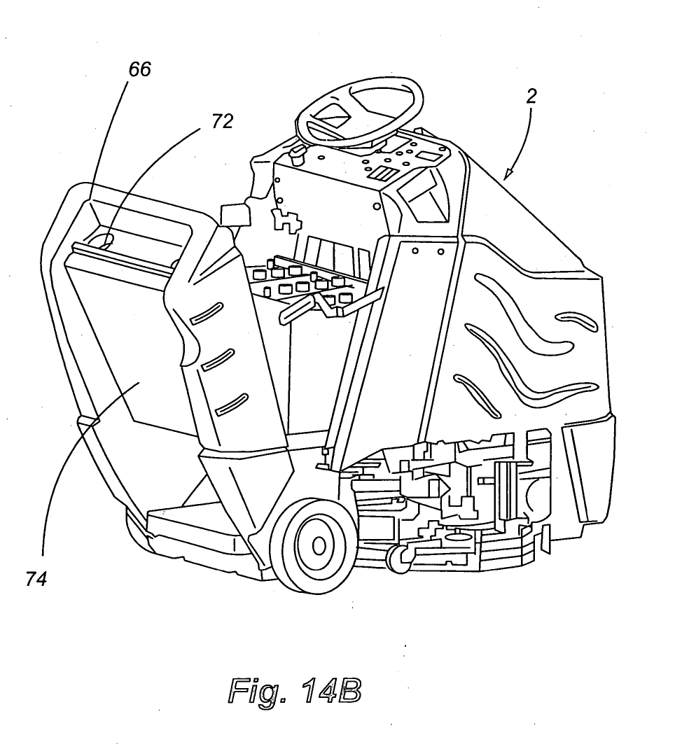

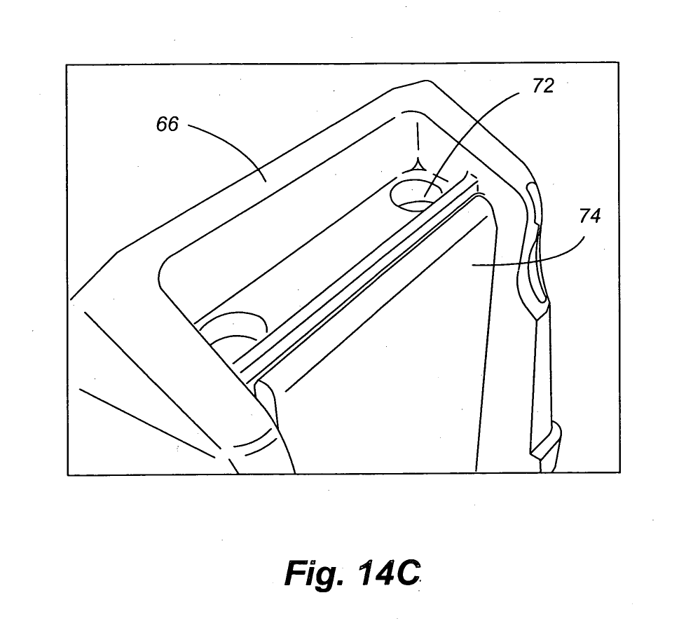

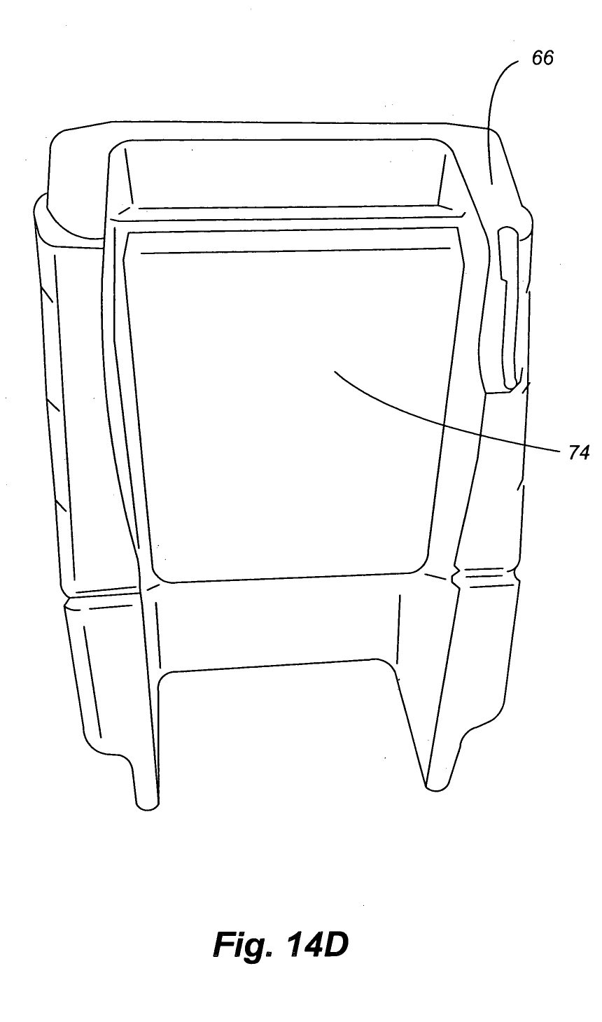

Referring now to FIG. 14, the rear portion of the floor treatment apparatus 2 is shown. More specifically, the rear of the apparatus 2 includes a removable cowling 66. The cowling 66 of the present invention is capable of selective rotation about an axis parallel to the rear axle of the apparatus 2. Alternatively, the rear cowling 66 may be completely removable. This aspect of the present invention provides the ability to access batteries 68 that may provide power to the apparatus 2. The batteries 68 may reside on a removable tray 70 that is slidingly engaged to the apparatus 2, thus providing easy access for maintenance. The tray 70 resides on tracks that interface with a plurality of wheels, bearings, etc. The tray also includes a locking feature that securedly maintains the batteries 68 inside the vehicle. The rear cowling 66 also includes other features, such as a cavity for securing various items and drink holders 72. A pad 74 may also be included that provides greater protection and comfort to the user.

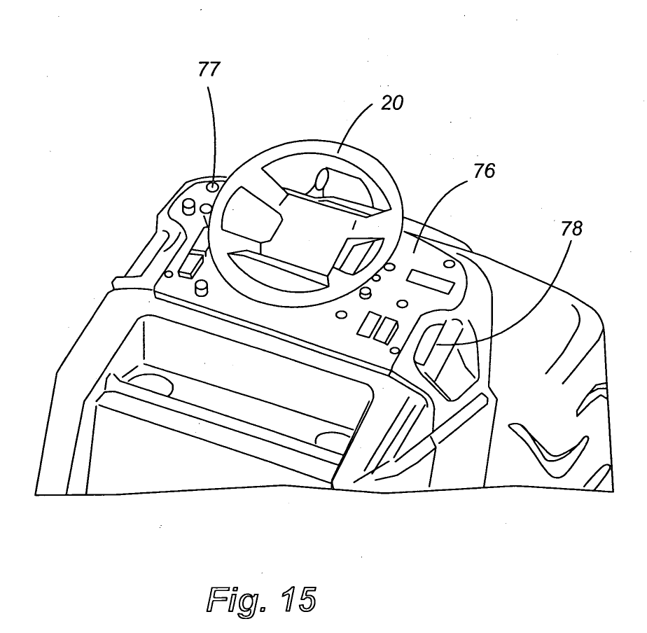

Referring now to FIG. 15, a control panel 76 and associated structure of one embodiment of the present invention is shown. Embodiments of the present invention include a control panel 76 that includes minimal fasteners 72 for interconnection to the floor treatment apparatus 2. That is, thumb screws, or similar type of fasteners may be included such that quick and easy removal of the control panel 76 may be achieved to facilitate repair.

Embodiments of the present invention also include hand grips 24 adjacent to the control panel 76 to provide support for the operator. More specifically, during tight turns the inertial forces acting upon an individual may cause an operator to fall. Hand grips 24, which may be integrated onto the chassis of the apparatus, will give the operator a place to hold onto the device for added comfort and provide an additional safety feature. In addition they provide support when operating control switches located adjacent to handle grip.

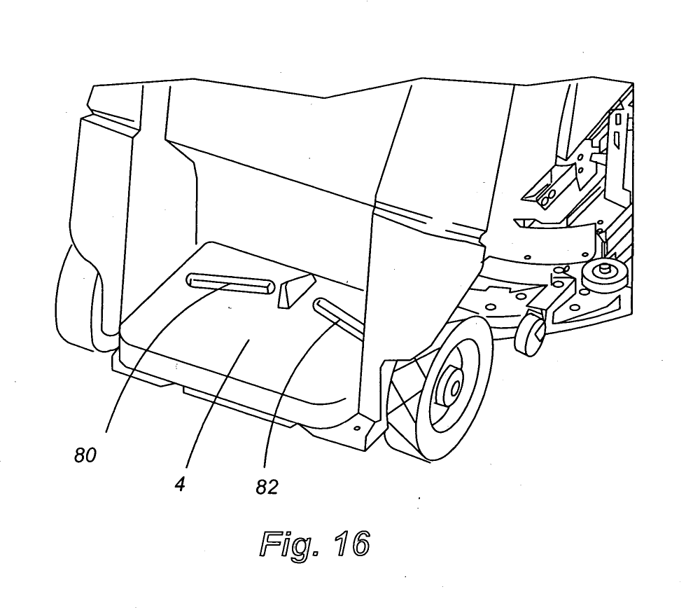

Referring now to FIG. 16, the platform 4 of one embodiment of the present invention is shown. More specifically, one embodiment of the present invention includes a platform 4 with an operator presence switch 80, a platform switch and a throttle 82. The platform 4 also may include a suspension system and be cushioned to increase operator comfort. In addition, the platform 4 may be foldable such that the envelop of the apparatus may be selectively reduced.

The operator presence switch 80 of one embodiment of the present invention is designed to act as a safety feature that interrupts the throttle pedal when not depressed. This ensures that the operator has both feet positioned on the platform when the machine is in use. Upon deactivation of the switch, for example if the operator removes a foot from the switch, a neutral mode may be engaged such that no power or forward or rearward motion of the device is possible. In addition, the operator presence switch 80 may ensure that sufficient weight is maintained on the platform at all times as a safety feature.

In the typical use, the platform switch is in operable connection with the platform, such that it is activated when the operator stands on the platform. The operator must then engage a reset device, preferably on the control panel, to initiate motion. The purpose of the platform switch and reset switch is to act as a safety feature such that the machine does not immediately move when the operator steps on to the peddle platform. Upon deactivation of the switch, for example if the operator steps from the apparatus, a neutral mode may be engaged such that no power and forward or rearward motion is possible.

The throttle 82 of some embodiments of the present invention is adapted to selectively increase or decrease the speed of the apparatus depending on the desires of the operator. More specifically, various speed ranges may be included: neutral, first, second, third, reverse, etc. (or slow, medium, fast, etc.). In some embodiments, cleaning operations are performed at slow speeds, while transportation from location to location is performed at higher speeds. When the operator sets the speed range to first, for example, the activation of the throttle 82 will propel the apparatus within that speed range, such that it can not transition from the first range to the second range without a manual shift of the range. Thus, embodiments of the present invention include a hand speed range selector, wherein the throttle 82 simply turns the desired speed range to an activated mode. The neutral mode may also be set by the operator, wherein no amount of throttle 82 engagement will increase the speed of the apparatus. In addition, as mentioned briefly above, when the operator removes his or her foot from the operator presence switch 80, the apparatus automatically disengages the throttle. One skilled in the art will appreciate however, that a throttle 82 may be provided that provides selective speed increments, such as employed on an automobile, without departing from the scope of the invention.

Embodiments of the present invention also include a braking mechanism. For example, when an operator removes his or her foot from the operator presence switch 80, throttle, or disengages the platform switch, a braking mechanism may be employed such that any motion of the apparatus automatically or gradually ceases. The braking mechanism may be electro mechanical, mechanical or hydraulic. Alternatively, the foot brake may be provided adjacent to the throttle 82 or operator presence switch 80 that provides the same halting capability. Further, hand or emergency brakes may be employed adjacent to the control panel of the apparatus.

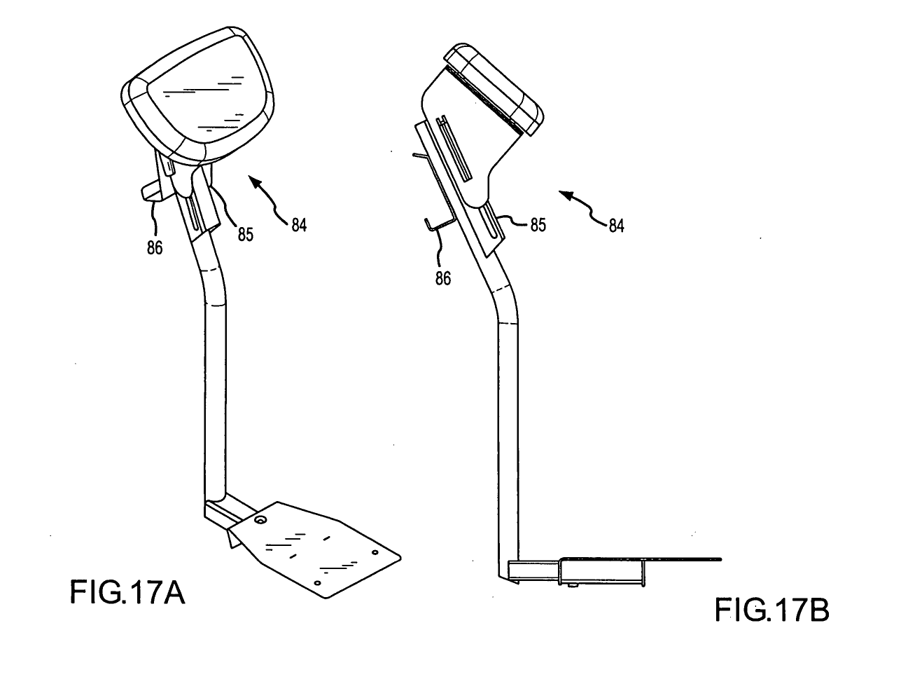

Referring now to FIG. 17, a seat 84 of one embodiment of the present invention is shown. More specifically, embodiments of the present invention include a selectively connectable seating device 84 for engagement with the chassis to increase the operator comfort. Seats 84 of some embodiments of the present invention are selectively adjustable 85, thus making them easy to accommodate any sized individual. In operation, a receiver hitch, or similar connection mechanism, is connected to the rear portion of the platform 4 and a mating device for interconnection to the receiver hitch, or other device, is provided on the seat 84. The seat 84 may also include a plurality of hooks, shelves, cup holders, etc. for the securement of cords, bags, or any other type of cleaning or comfort related item. Further, the receiver hitch may be used when the seat 84 is engaged or not engaged, for example, to transport other items such as a supplemental wheeled device that may accommodate extra power sources, cleaning supplies, tanks, etc.

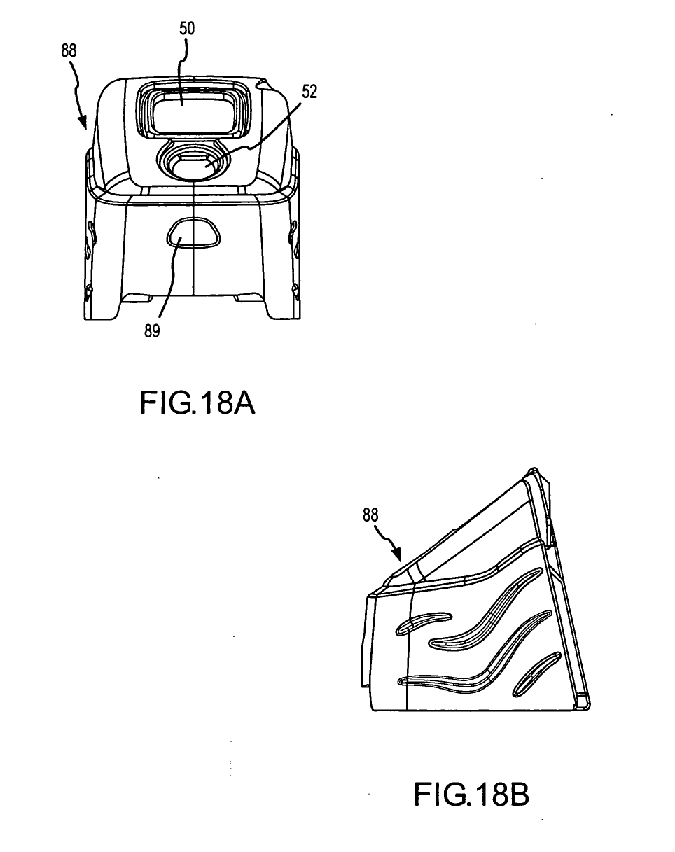

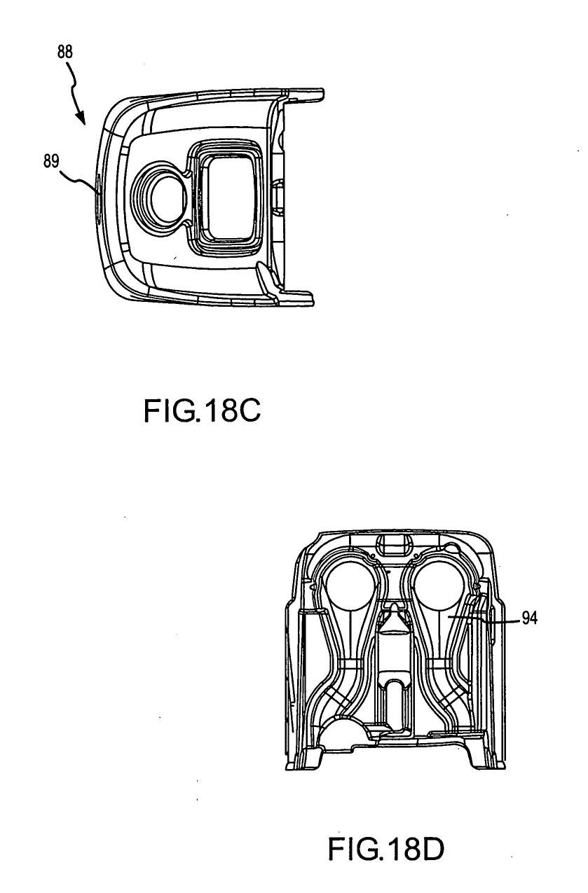

Referring now to FIG. 18, a tank 50 of one embodiment of the present invention is shown. Some embodiments of the present invention include a tank 50 that is equipped with a plurality of lights 80 and/or horns that facilitate cleaning and/or act as additional safety features. Alternatively, lights may be integrated into bumpers positioned adjacent to the tank 50 or on the sides of the apparatus.

Although not shown, a filter may be provided in fluid communication with the fluid pump. This filter is designed to capture any debris that may adversely affect the operation of the pump. Unfortunately on many cleaning machines, the filter is placed in a hard to access location, such that repair or monitoring thereof is very difficult. Thus, one embodiment of the present invention includes a filter that is situated on the outer surface of the cowling, perhaps on the control panel. Thus, the operator has ample opportunity to monitor the integrity of the filter and make quick repairs when necessary.

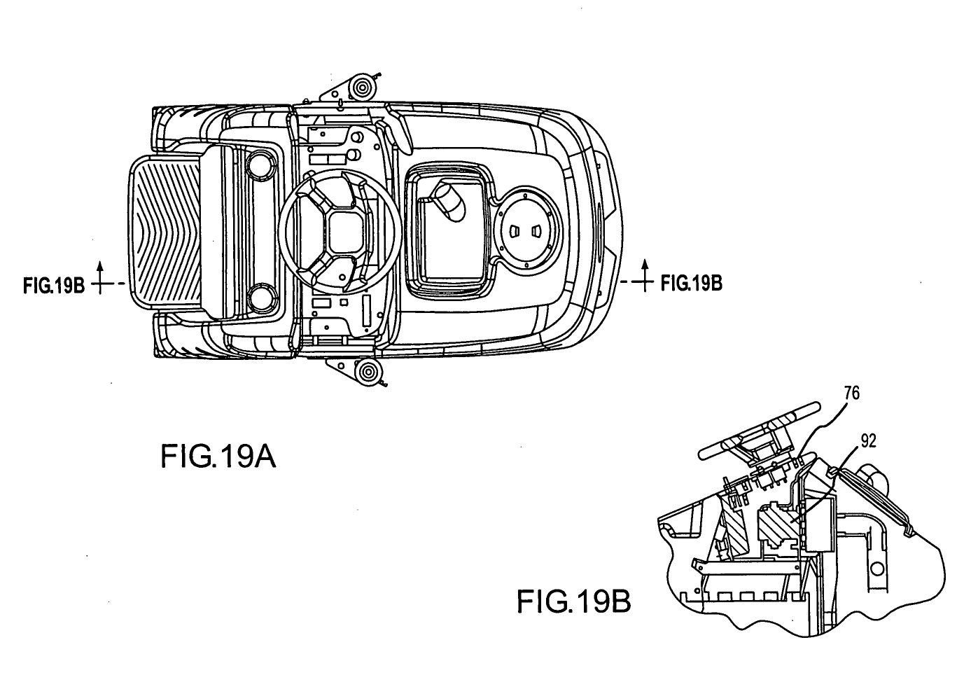

Referring now to FIG. 19, a vacuum fan 92 which is connected to the front cowling 88 of one embodiment of the present invention is shown. More specifically, a vacuum fan 92 provides suction to remove debris filled fluids from the floor. The fan 92 is preferably situated under the control panel 76 of the vehicle, such that the intake cooling air that is drawn in by the vacuum fan 92 is channeled adjacent to the control panel 76 to cool componentry associated therewith.

In addition, the tank 50 may be made out of a formable material such that exhaust channels 94 may be machined or molded into the tank 50. The channels 94 direct the exhaust air from the vacuum 92 to an exit muffler of the apparatus. The channels 94 also act as a baffle to remove noise energy from the exhaust gases, thus making the entire system more quiet.

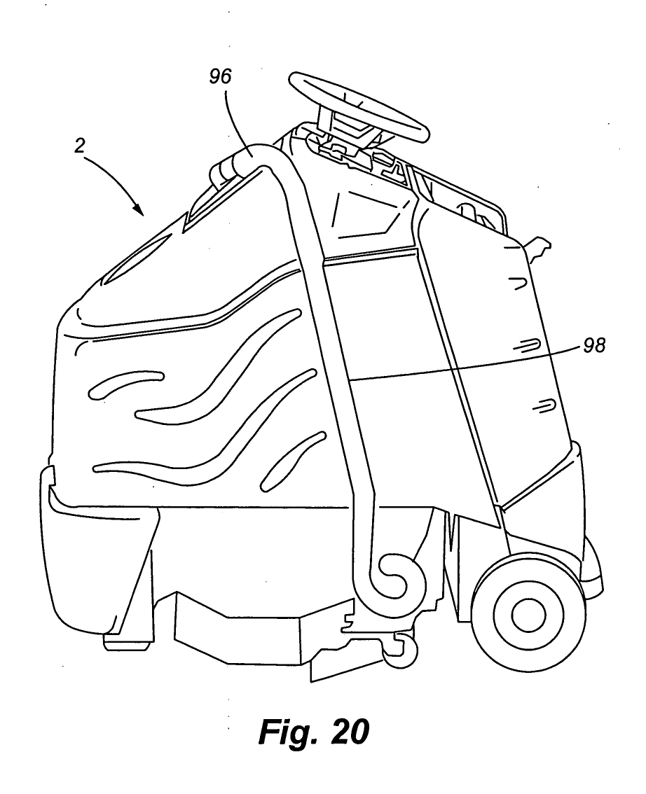

Referring now to FIG. 20, a waste fluid return hose 96 is shown. More specifically, one embodiment of the present invention decreases its profile by inserting the waste water hose 96 into a hose channel 98 that is integrated into the outside surface of the apparatus 2. The hose 96 being situated on the outside also has the added advantage of making it very accessible, such that it can be removed and inspected for clogs or breaches.

Further, some embodiments of the present invention are provided with tip over stops 100 adjacent to the front corners of the apparatus. The stops 100 may be replaceable and ensure that the apparatus does not tip over during tight turns. The tip over stops 100 are generally constructed out of a material that is harmless to flooring, such as Teflon, silicone, rubber, plastic, etc. In addition, one skilled in the art will appreciate that rollers may be employed that are situated a predetermined distance from the floor to perform the same function.

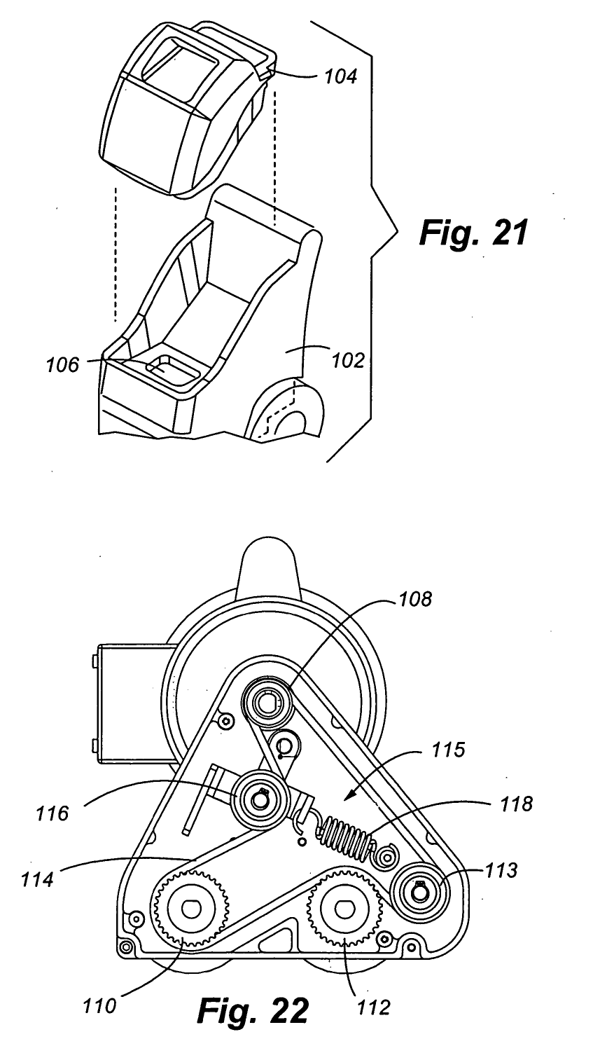

Referring now to FIG. 21, a base solution tank 102 and fluid recovery tank 104 are shown. In one embodiment, the interface between the solution tank 102 and recovery tank 104 is designed such that during use the recovery tank 104 does not slip out of place. The recovery tank 104 is partially nested within the solution tank 102 in order to not use excess space. This compact design increases the ability to store the cleaning apparatus 2 in compact spaces. Additionally, a unique tank orifice 106 is provided on the base solution tank 102. Unlike prior cleaning devices, the tank orifice 106 has no upward extending lips or edges. When an operator 6 wishes to empty the dirty fluid in the fluid recovery tank 104 usually he or she will refill the recovery tank 104 with a new supply of clean fluid, like water, to refill the solution tank 102. Because the tank orifice 106 has no upward extending edges, an operator 6 can easily pour the fresh water into the solution tank 2 without worrying about excess spillage or trying to hit a very small target. Rather, the funnel design of the tank interface facilitates the pouring of clean water, or other base fluid, into the solution tank where the operator 6 is allowed to pour the clean water virtually anywhere close to the orifice 106.

Referring now to FIG. 22 a single motor drive design utilizing a single belt will be discussed in accordance with embodiments of the present invention. In one embodiment, a single motor is used to power two brushes. The drive design comprises a motor pulley 108, a first brush pulley 110, a second brush pulley 112, a third pulley 113, and a timing belt 114. Preferably, the timing belt 114 is a two sided timing belt that interconnects the motor pulley 108 with the first and second brush pulleys 110 and 112 and the third pulley 113. Additionally, in one embodiment, the drive design includes a tensioning member 115. The tensioning member 115 may comprise an idler pulley 116 and a spring and ratchet member 118 in one embodiment. The tensioning member 115 is used to apply a substantially constant force on the timing belt 114 such that an operator 6 of the apparatus 2 does not have to manually set the tension on the belt. Preferably, the idler puller 116 has the timing belt 114 wrap on the same side of the pulley 116 as the ratchet and spring member 118. This allows the spring to be in tension, and minimizes the amount of space used by the entire drive assembly. The tensioning member 115 may be spring loaded on the slack side of the belt 114. Of course, the tensioning member 115 may be oriented such that the spring is in compression and the belt 114 wraps around the outside of the idler pulley 116.

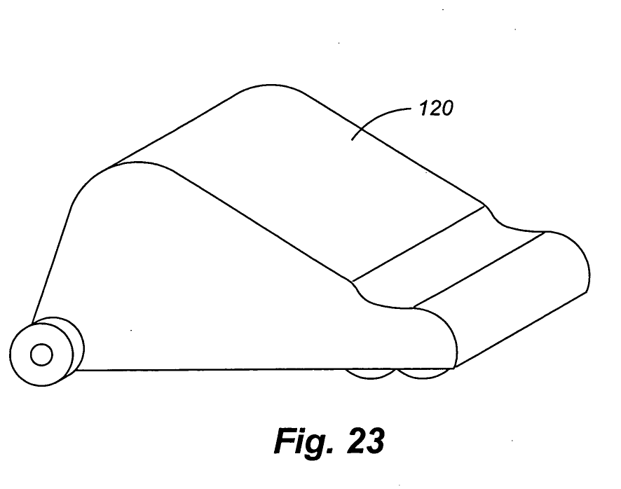

With reference now to FIG. 23 a wide area vacuum 120 attachment, for use with the cleaning apparatus 2 will be discussed in accordance with one embodiment of the present invention. Specifically, the wide area vacuum 120 is a wet or dry vacuum that utilizes a dual brush system in one embodiment of the present invention. In accordance with embodiments of the present invention, the dual bush system may be driven by the single motor drive assembly described above. Alternatively, the brushes of the wide area vacuum 120 may each be independently driven by their own dedicated motors. The wide area vacuum 120 attachment is operable to be selectively connected and disconnected from the chassis 8 such that other cleaning apparatus may be employed. The length of the rollers utilized by the wide area vacuum may vary depending on the application and size of cleaning area. In one embodiment the length of the brushes can be between about 16″ to about 30″. More preferably the brushes are between about 24″ and about 28″, with a most preferred embodiment employing brushes that are 26″ long. Of course, depending upon the application, different brush lengths may be preferred. As can be appreciated, the wide area vacuum housing may be of various sizes according to the desired brush size. However, the vacuum housing can be designed such that brushes of various sizes can be used with a single vacuum housing.

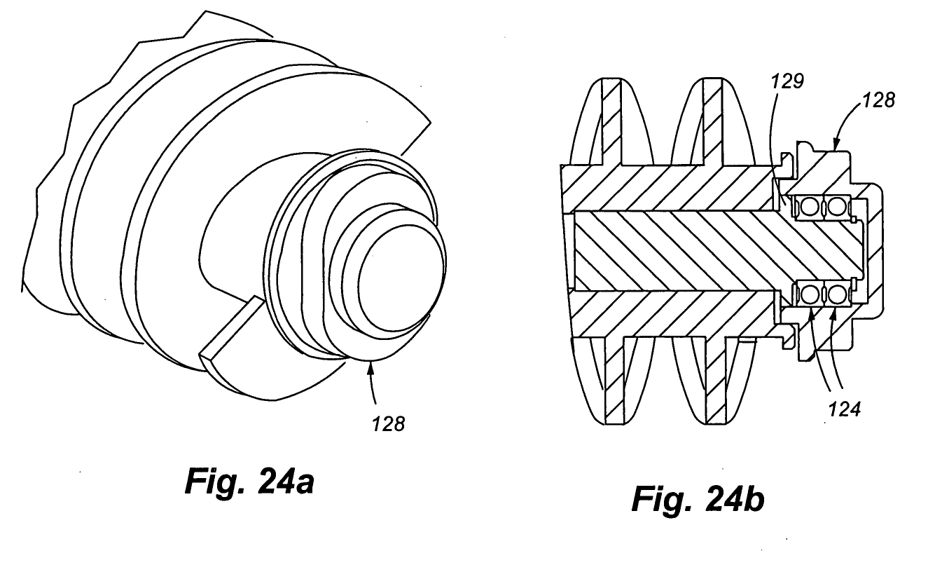

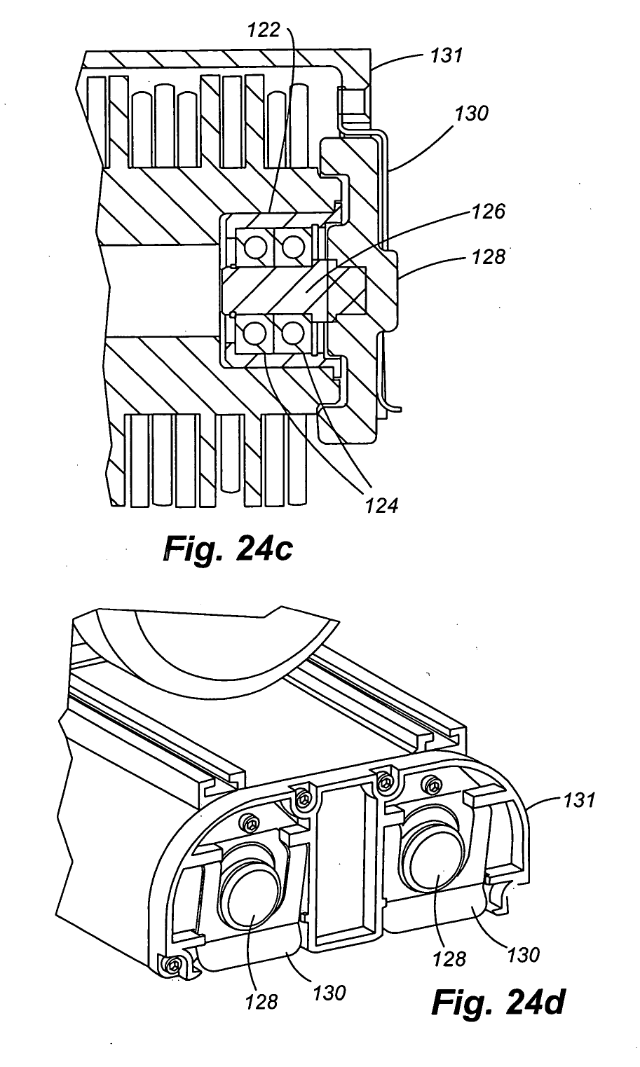

Referring now to FIG. 24a-d, a mounting system for use with cylindrical brushes 121 will be described in accordance with embodiments of the present invention. Often hair and other unwanted debris manages to get wrapped around the bearings and spindle of a brush 121. The bearing housing is especially sensitive to unwanted debris. If too much debris infiltrates the bearing housing, the brushes tend to stop working properly. To mitigate these problems, the present invention, in one embodiment, uses a bearing protection system. The mounting system for the non-drive side of the brush 121 comprises a bearing insert 122, one or more bearings 124, a shaft 126, an end cap 128, and a spring clip 130. The bearing(s) 124 may be any type of bearing including a ball bearing, a roller bearing, a needle bearing, a ball thrust bearing, a roller thrust bearing, and a tapered roller thrust bearing. In use, the bearing(s) 124 sits around the shaft 126 and is mounted to the end cap 128. The shaft 126 is inserted into the end cap 128. The insert 122 then closes the opening of the end cap 128 and the entire assembly is attached to the inner wall of a brush housing 131 with the spring clip 130. The insert 122 receives the brush 121 and the brush 121 rotates about the shaft 126. The bearing(s) 124 facilitate a smooth rotation of the shaft 126. The brush 121 is held in the insert 122 by a flange 129. The flange 129 helps ensure that the brush 121 does not pull away from or push into the mounting. The insert 122 along with the end cap 128, in one embodiment of the present invention, act to create a longer distance that debris has to travel before it reaches the bearing(s) 124 and/or spindle housing. The system helps prevent hair or other unwanted debris from collecting around the spindle or bearings which would otherwise inhibit the rotation of the brush 121.

As can be seen in FIG. 24d, the spring clip 130 provide for easy access to the brush 121. While in operation the spring clip 130 engages the brush end cap 128 such that the brush 121 does not slip or fall out. The brush 121 is free to rotate within the end cap 128, with the assistance of the bearing(s) 124. An operator is able to gain easy access to the brush 121 since the spring clip 130 is openly available. The brush 121 can be easily inserted/removed from the housing by releasing the spring clip and sliding the brush 121 in/out of the housing 131.

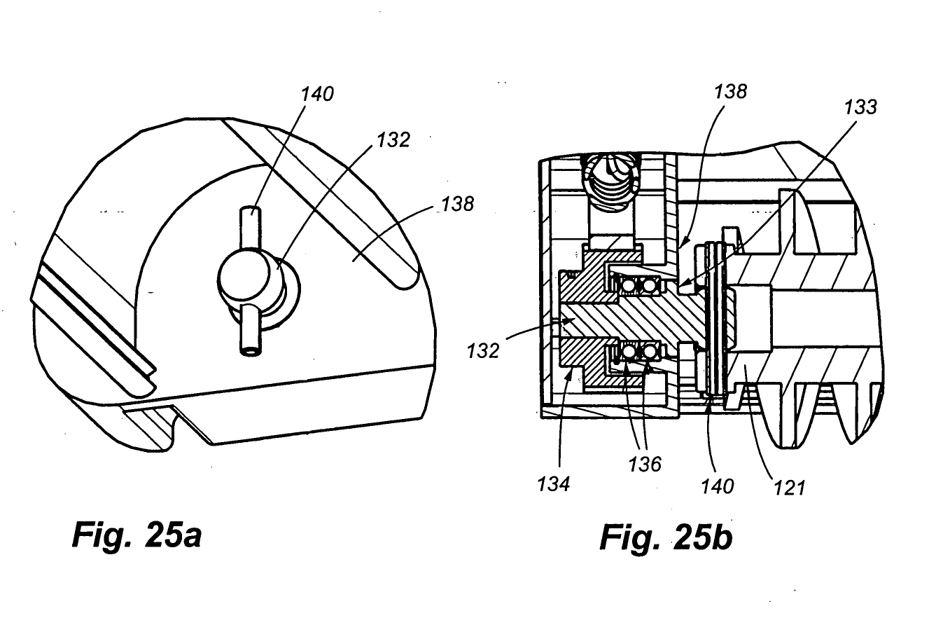

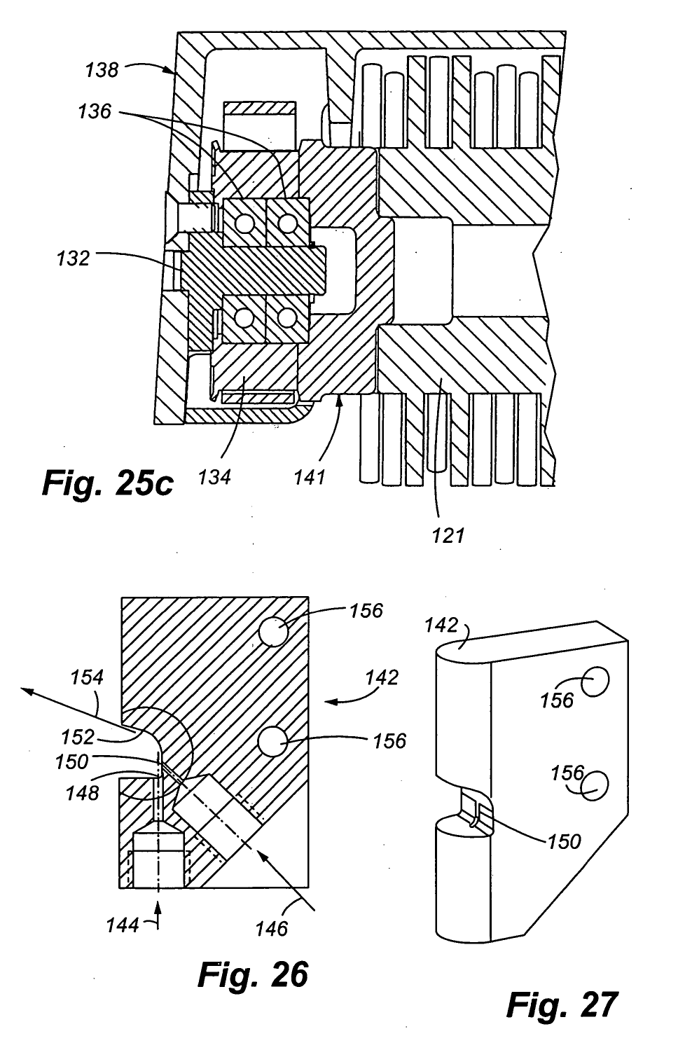

Referring now to FIG. 25a-c, the drive side mounting system for a cylindrical cleaning brush 121 used in accordance with one embodiment of the present invention will be discussed. The drive side assembly includes, in one embodiment, a drive shaft 132 having a flange 133, a brush pulley 134, one or more bearings 136, and a drive pin 140. The brush 121 is typically driven by a belt traversing the brush pulley 134 and rotating the drive shaft 132. The bearing(s) 136 are mounted to a drive housing 138. The drive shaft 132 is inserted into the bearing(s) 136 and brush pulley 134. The brush pulley 134 rotates the drive shaft 132, which in turn rotates within the bearing(s) 136. The drive shaft 132 is typically maintained in the housing 138 by a flange 133. The flange 133 helps ensure that the brush 121 does not pull away from or push into the housing 138. As can be seen in FIG. 25c a drive hub 141 may be used in place of the flange 133 to keep the brush 121 substantially laterally fixed with respect to the housing 138. The brush 121 is then connected to the assembly by the use of a drive pin 140. The pins 140 may also act to hold the entire assembly together. A tight fit between the drive shaft 132, its flange 141, and the drive housing 138 helps protect the bearing(s) 136 from unwanted debris that is agitated by the brush during cleaning.

With reference to FIGS. 26 and 27 a spray nozzle 142 will be discussed in accordance with embodiments of the present invention. The spray nozzle in one embodiment includes an insert for base fluid (water) flow 144 and an insert for cleaning solution flow 146. There may also be attachment points 156 provide on the nozzle to provide a way of attaching the nozzle to the cleaning apparatus 2. Water and/or other fluids are pressurized and forced through an orifice 148. The size of the first orifice can vary depending on the amount of water that is required for cleaning. It is understood, that the flow rate of the fluid can also be controlled by metering valves placed upstream of the insertion point on the nozzle. As fluid is ejected from the orifice 148 it passes past a siphon point 150. The siphon point 150 is the opening where the cleaning solution is allowed to exit the line. The velocity of the water crossing the opening of the cleaning solution acts to create a siphon and initiates a flow of the cleaning solution. The nozzle 142 design creates a siphon for the cleaning solution and the two solutions are mixed at their point of contact. The mixed stream of fluid continues until it strikes a dispersion surface 152. The dispersion surface 152 is designed, in one embodiment, to produce a wide angle flat spray pattern and the solution is then applied to the surface to be cleaned.

One skilled in the art will appreciate that the cleaning solution does not need to be pumped or supplied to the nozzle at any pressure. Rather, the passing of the fluid across the opening of the cleaning solution creates a siphon and cleaning solution is pulled from its source rather than pushed with a pump upstream of the nozzle. Additionally, the ratio of cleaning solution to base fluid can be controlled by the adjustment of the pressure at which the base fluid is supplied or can be controlled by changing the cleaning solution orifice size. An inventive aspect of nozzle, in accordance with embodiments of the invention, is that two solutions are mixed at their point of contact and application with only one of the solutions being under pressure before it is passed through the nozzle. The need for separate mixing mechanisms and application mechanisms is obviated. In a further embodiment, the cleaning solution can be the pressurized fluid and the base solution may be siphoned from its reservoir. Furthermore, multiple cleaning solutions can be mixed using similar embodiments of the present invention.

It can also be appreciated that the nozzle 142 can be used in conjunction with a larger cleaning apparatus 2 or may alternatively be used alone. For example, the nozzle 142 may be placed on the end of a cleaning wand or the like for remote cleaning of walls and other surfaces without having to use a larger cleaning apparatus.

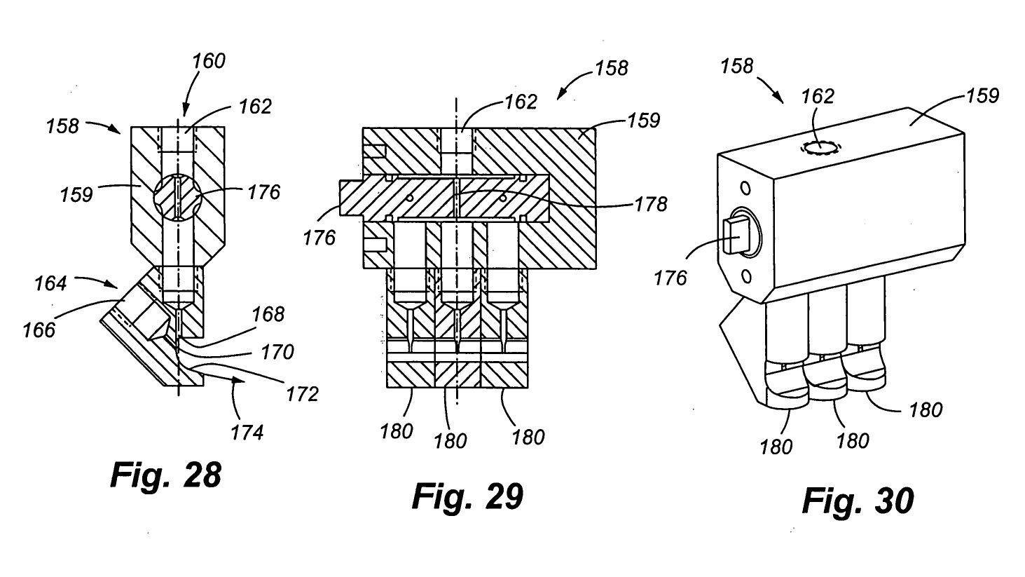

It can also be appreciated that a plurality of nozzles can be used together to form a mixing block 158 as will be described in accordance with embodiments of the present invention with reference to FIGS. 28-30. The mixing block 158, provides a way to combine a primary flow of a base fluid 160 with one or more of several cleaning solutions 164. In one embodiment, the mixing block 158 includes a valve housing 159, a base solution opening 162, a plurality of cleaning solution openings 166, a plurality of base fluid exit orifices 168, a plurality of siphon points 170, a plurality of dispersion surfaces 172, a selector 176, a selection valve, and a plurality of nozzles 180. A base fluid, like water, is pressurized and enters the mixing block 158 at the base fluid opening 162. Thereafter, the water passes through the selection valve 178. The selection valve 178 is housed in the valve housing 159 and selectively passes the water to one or more of a plurality of nozzles 180. The selection valve is actuated by a selector 176. The selector 176 can be an electronic or mechanical switch or other type of actuating device that provides a way to select one or more of the cleaning solutions at various ratios. The water is ejected past an orifice 170 for a cleaning solution as described above and the water is mixed with that cleaning solution. The water can be passed through multiple exit orifices 168 simultaneously or may be sent to only one orifice 168. This enables the mixing block 158 to create a large number of different mixed solutions having different ratios of cleaning solution(s) to the water. As described above, cleaning solution may be the fluid that is pressurized and passed over the siphon point and the water may be in one of the nozzles 180.

Use of the mixing block 158 in accordance with embodiments of the present invention allows an operator to easily change the cleaning solution as the situation dictates. For example, if during a first part of the day a carpet is to be cleaned, a combination of carpet cleaning solution with water can be mixed in the mixing block 158. Then with the simple use of the selector 176 a hard floor surface cleaner can be mixed with the water and the cleaning apparatus 2 can be used to clean hard floor surfaces. Additionally, if the surface is excessively dirty, the operator can add a more aggressive cleaner to the solution mix without having to go to a great deal of trouble. The selector 176 may also be used to change between cleaning modes. For example, if the selector 176 is in a first position, the cleaner and water may be mixed for a deep cleaning mode. Then the selector 176 can be changed into a second position. When in this position the cleaner and water may be mixed to provide for an interim clean, instead of a deep clean. Further still, a third position may allow the cleaner to create a mixture for a light cleaning. The operator simply has to use the selector 176 to change the kind and/or quantity of cleaning solution used.

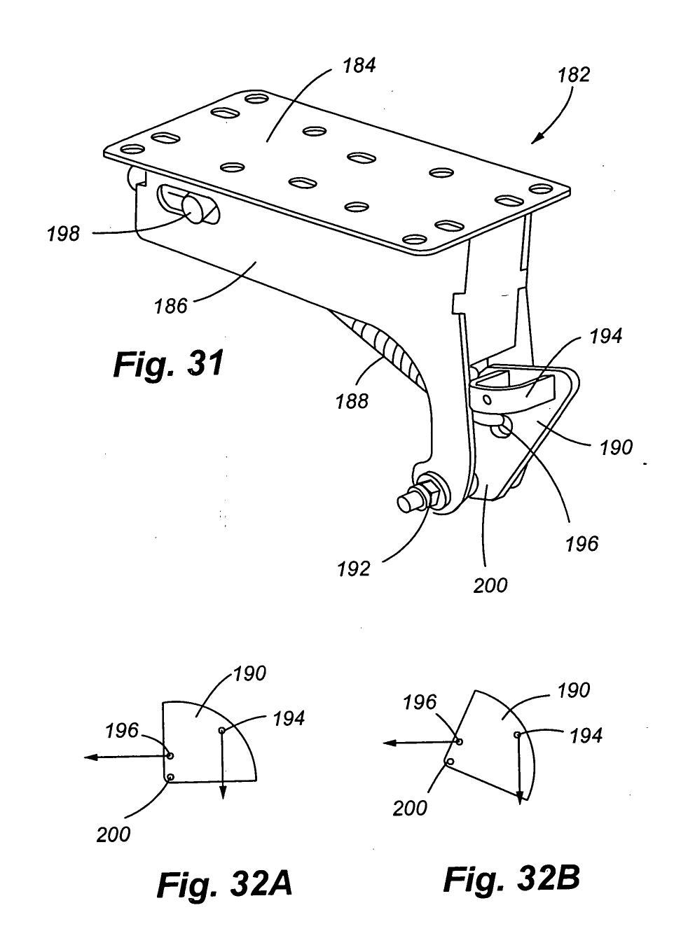

Referring now to FIGS. 31-33 a counter balance mechanism 182 will be described in accordance with embodiments of the present invention. The counter balance mechanism 182 enables a floor treatment component like a vacuum deck, set of brushes, scrubber, squeegee, etc. to pass over a non-smooth surface while applying about the same amount of force to all portions of the floor. In one embodiment the counter balance mechanism 182 includes, a base plate 184, a set of side plates 186, a spring 188, an interconnection member 190, and a pivot bolt 192. The interconnection member 190 includes a deck attachment point 194, a spring attachment point 196 and a pivot point 200. The base plate 184 bolts or is otherwise attached to the bottom surface of the chassis 10. The two side plates 186 are connected to the base plate by screws, bolts, or it is permanently welded in place thereto. The interconnection member 190 is connected to the side plates 186 by a bolt or screw 192. The bolt 192 passes through the interconnection member 190 at its pivot point 200. The interconnection member 190 is free to pivot relative to the side plates around this pivot point 200. The spring 188 connects (in tensions) between a spring adjustment member 198, which can be a pin, bolt or the like, and the interconnection member 190. Generally, the tension of the spring 188 can be adjusted by moving the spring adjustment member 198 forward or backward relative to the side plates 186. A deck 202 is connected to the interconnection member 190 at the deck attachment point 194. The deck 202 can be attached directly to the deck attachment point 194 or may be connected to the deck attachment point by way of an attachment member 204, for instance a chain, bar, rope or other type of force applying member.

As can be seen in FIG. 32, in one embodiment, the floor treatment component (deck) 202 applies a downward force on the interconnection member 190 at the deck attachment point 194 due to it's weight. The spring 188 in tension applies a force that counters the moment created by the weight of the deck 202. These opposite moments act to balance the load applied by the deck 202 to the floor. Essentially, if the contour of the surfaces changes the force applied by the deck 202 changes as well. In reaction to this change in force the interconnection member 190 rotates about it's pivot point 200 and the force applied by the spring changes. More relevant than the forces changing that are applied to the interconnection member is that the moment arms of each force change as the contour of the surface changes. When the interconnection member rotates as seen in FIG. 32, the moment arm of the force applied by the deck 202 increases whereas the moment arm of the force applied by the spring 188 decreases. These changing moment arms about the pivot point 200 of the interconnection member 190 help the deck maintain a substantially constant applied force relative to the ground. The design also allows the deck 202 to recess toward the chassis 8 when the entire floor treating apparatus is lifted and/or tilted for storage or the like.

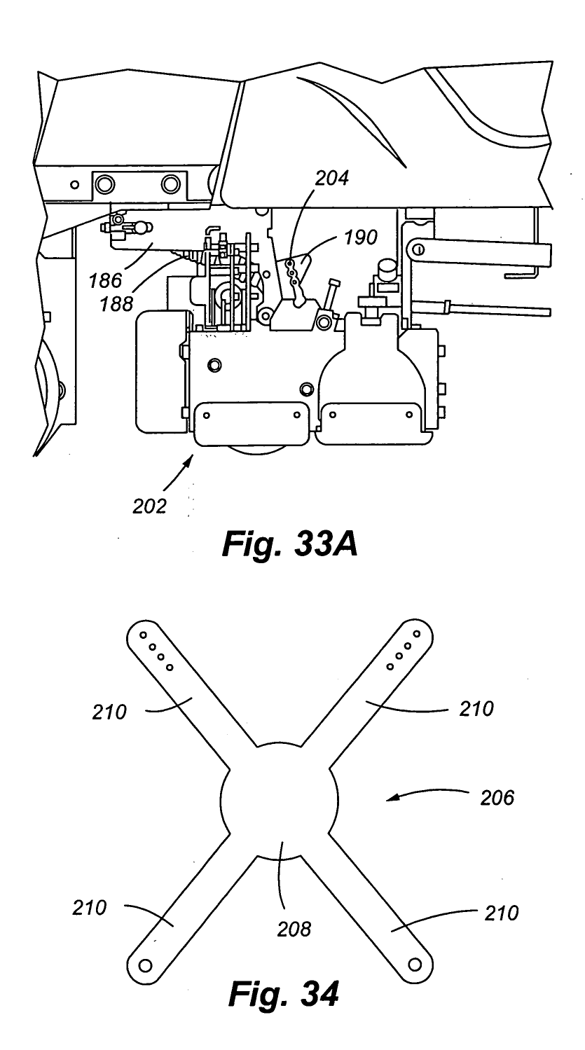

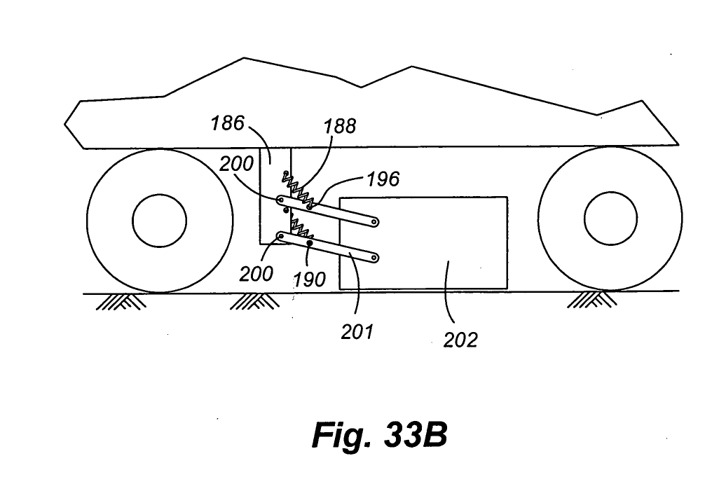

Alternative configurations of the counter balance mechanism 182 are possible. For example, the deck may be directly connected to the machine by one or a set of arms as can be seen in FIG. 33B. In this particular configuration, the side plates 186 are attached directly to the chassis 8. Instead of utilizing a single pivot point and a single spring as depicted in FIG. 33A a set of linking members 201 connect the deck 202 to the side plates 186 at pivot points 200. These pivot points correspond to the single pivot point 200 discussed above, but since there are two linking members 201, each member has it's own pivot point 200. The deck 202 applies a generally downward force on the linking members 201 at the ends opposite the pivot points 200. Springs 188 are connected to the side plates 186. The springs 188 also connect to the linking members 201 at spring attachment points 196. The springs 188 are in tension in order to apply a generally upward force to the linking members 201. This particular configuration enables the deck 202 to pass across the surface while applying a relatively constant downward force on the surface. As can be appreciated by one of skill in the art, additional configurations can be envisioned. For example, the springs 188 may be replaced by weights attached to a cable and pulley system. The weights may apply the same general upward for as the springs 188 do through a series of cables and pulleys. The dual linking arms described may be replaced with a single linking arm depending on the size and orientation of chassis 8 and deck 202. The counter balance mechanism 182 can also be designed in a number of other fashions such that the moment about the pivot point created by the deck's weight are countered by opposite moments about the pivot point created by another force from e.g. a spring, cable, bar, and/or chain. As the counter balance mechanism 182 moves in reaction to an uneven surface, excessive forces are not applied by the deck to the surface due to the changing moment arms about the pivot point.

With reference to FIG. 34 a retention member 206 for use with the cleaning apparatus 2 is described in accordance with embodiments of the present invention. In one embodiment, the retention member 206 includes a body 208 and connection arms 210. The body 208 serves as a central connection point for all of the connection arms 210. The retention member 206 is operable to interface with the chassis 8 of the cleaning apparatus 2 in order to form a nesting design that allows any type of container (e.g., hand pump and sprayer, fluid tank, trash can, etc.) to be conveniently attached to the cleaning apparatus 2 for ease of transportation and operation. In one embodiment, two of the connection arms 210 can be fastened either fixedly or selectively to the chassis 8 by a connection member like snap fasteners, buckles, glue, hook and loop material, and the like. The other two connection arms 210 have a plurality of holes incorporated into the material to selectively secure the other arms to the chassis 8. To insert a container the operator only needs to release the two selectively secured arms 210 and the container can be placed in the pocket formed by the retention member 206. The arms 210 can then be reattached to the chassis 8 to ensure safe travel of the container. As can be appreciated, a number of configurations are possible that would provide the same service as the one described above. Namely, a different number of connection arms 210 could be used and a body 208 may not be required. Many configurations of connection members to create a container retention member 206 is envisioned.

Referring now to FIGS. 1-34, a manner of making the present invention is shown and described herein. As has been explained, the present invention is generally similar to the floor treatment devices used in the art. However, unlike many prior art devices, the present invention provides a location for which the operator can stand or sit, thus enabling him or her to more efficiently perform their tasks. In addition, instead of using brute strength to perform the task of steering the cleaning device, a steering mechanism and associated hardware are provided to aid in the smooth transition from one direction to another. Also, the present invention device has a compact profile and mechanism which allows for 360° cleaning of tight spaces. Further, to construct the remote control version of the system, software that is known in the art may be installed in the chassis 8 to allow for the system to be either remotely controlled or learn the cleaning surface as it operates. In addition, a series of cameras may be interconnected to the chassis 8 to provide remote viewing to an operator offsite.

While various embodiment of the present invention have been described in detail, it is apparent that modifications and alterations of those embodiments will occur to those skilled in the art. However, it is to be expressly understood that such modifications and alterations are within the scope and spirit of the present invention, as set forth in the following claims.

What is claimed is:

1. A spray nozzle for use with a surface cleaning apparatus, comprising:
a primary solution line having an inlet for receiving a primary solution and an exit orifice;
at least one secondary solution exit orifice associated with at least one secondary solution; and
wherein the primary solution is forced through the primary solution line, out the primary solution exit orifice and past the at least one secondary solution exit orifice thereby inducing flow of the at least one secondary solution out of the at least one secondary solution exit orifice.

2. The spray nozzle of claim 1, wherein in response to passing the primary solution past the at least one secondary solution exit orifice, the primary solution and at least one secondary solution are mixed to form a mixed solution.

3. The spray nozzle of claim 2, wherein the mixed solution has a predetermined ratio of the primary solution and the at least one secondary solution.

4. The spray nozzle of claim 2, further comprising a dispersion surface, wherein the mixed solution strikes the dispersion surface prior to being applied to a surface to be cleaned.

5. The spray nozzle of claim 1, further comprising a selection valve that controls the amount of primary solution that passes across each of the at least one secondary solution exit orifices.

6. The spray nozzle of claim 5, further comprising a selector that is coupled to the selection valve, wherein the selector may be adjusted by a user of the spray nozzle.

7. The spray nozzle of claim 6, wherein the selector is an electronic switch.

8. The spray nozzle of claim 6, wherein the selector is a mechanical switch.

9. The spray nozzle of claim 1, wherein at least one of the primary solution and the at least one secondary solution includes water.

10. The spray nozzle of claim 1, further comprising a metering valve that controls the amount of primary solution that enters the primary solution line.

11. The spray nozzle of claim 1, wherein the at least one secondary solution exit orifice is open to atmospheric pressure.

12. A spray nozzle for use with a surface cleaning apparatus, comprising:
a primary solution line having an inlet and an exit orifice, wherein a primary solution under pressure is forced through the line out the exit orifice;
a selection chamber having an inlet in fluid communication with the primary solution line exit orifice and a plurality of exit orifices; and
a plurality of secondary solution exit orifices adapted to transmit a plurality of secondary solutions, wherein the selection chamber controls an amount of primary solution passed across each of the plurality of secondary solution exit orifices thereby inducing a flow of at least one of the plurality of secondary solutions.

13. The spray nozzle of claim 12, wherein the amount of primary solution passed across a first of the plurality of secondary solution exit orifices is not the same as the amount of primary solution passed across a second of the plurality of secondary solution exit orifices.

14. The spray nozzle of claim 12, wherein each of the plurality of secondary solutions are a different solution.

15. The spray nozzle of claim 14, wherein in response to passing the primary solution past the at least one of the plurality of secondary solution exit orifices, the primary solution and the at least one of the plurality of secondary solutions are mixed to form a mixed solution.

16. The spray nozzle of claim 15, further comprising a dispersion surface, wherein the mixed solution strikes the dispersion surface prior to being applied to a surface to be cleaned.

17. The spray nozzle of claim 16, wherein the mixed solution passes from the plurality of secondary solution exit orifices to the dispersion surface through open air.

18. The spray nozzle of claim 16, wherein the dispersion surface creates a wide angle flat spray pattern of the mixed solution.

19. The spray nozzle of claim 12, wherein the flow of the at least one of the plurality of secondary solutions each depend upon the amount of primary solution passed across each of the plurality of secondary exit orifices.

20. The spray nozzle of claim 12, wherein the primary solution is primarily comprised of water.

21. The spray nozzle of claim 15, further comprising a selector that controls a ratio of the primary solution and the at least one of the plurality of secondary solutions in the mixed solution.

22. The spray nozzle of claim 12, wherein the plurality of secondary solution exit orifices are open to atmospheric pressure.

23. A spray nozzle for use with a surface cleaning apparatus, comprising:
a means for distributing a primary solution;
a means for distributing a secondary solution;
a means for forcing the primary solution out of the means for distributing the primary solution and past the means for distributing the secondary solution such that the secondary solution is siphoned out of the means for distributing the secondary solution; and
wherein the means for distributing the primary and secondary solutions are open to atmospheric pressure.

24. The spray nozzle of claim 23, wherein in response to passing the primary solution past the means for distributing the secondary solution, the primary and secondary solutions are mixed to form a mixed solution.

25. The spray nozzle of claim 24, further comprising a means for dispersing, wherein the mixed solution strikes the means for dispersing prior to being applied to a surface to be cleaned.

26. The spray nozzle of claim 24, further comprising a means for controlling, wherein a ratio of primary solution and secondary solution in the mixed solution is controlled by the means for controlling.
